(19) United States
(12) Patent Application Publication
(10) Pub. No.: US 2019/0082664 A1
(43) Pub. Date: Mar. 21, 2019

(54) HUMANIZED MOUSE MODEL FOR CANCER METASTASIS

(71) Applicant: The Jackson Laboratory, Bar Harbor, ME (US)

(72) Inventors: Anna Karolina Palucka, Avon, CT (US); Jacques Banchereau, Farmington, CT (US); Chun I. Yu, New Britain, CT (US)

(73) Assignee: The Jackson Laboratory, Bar Harbor, ME (US)

(21) Appl. No.: 16/131,256

(22) Filed: Sep. 14, 2018

Related U.S. Application Data
(60) Provisional application No. 62/765,257, filed on Aug. 20, 2018.
(60) Provisional application No. 62/558,788, filed on Sep. 14, 2017.

(51) Int. Cl. A01K 67/027; G01N 33/50

(52) U.S. Cl. CPC A01K 67/0278; G01N 33/5023; A01K 2267/0331; G01N 2333/70596; A01K 2227/105

(57) ABSTRACT

The present disclosure provides, in some aspects, a humanized mouse (NSG™-SGM3), engrafted with CD34+ hematopoietic progenitor cells and human metastatic melanoma cells, which surprisingly promotes secondary metastatic colonization, modeling the interplay between human tumors and the human immune system, and thus enabling mechanistic and pre-clinical studies for the development of novel treatment strategies targeting human-specific molecular pathways controlling melanoma dissemination.

### RELATED APPLICATIONS

This application claims the benefit under 35 U.S.C. § 119(e) of U.S. provisional application No. 62/558,788, filed Sep. 14, 2017, and U.S. provisional application Ser. No. ______, filed Aug. 20, 2018, each of which is entitled Humanized Mouse Models for Cancer Metastasis and each of which is incorporated by reference herein in its entirety.

### BACKGROUND

Cancer is a leading cause of death in the United States. Information from the U.S. National Cancer Institute's Surveillance Epidemiology and End Results (SEER) database showed that 1 out of every 2 people will be diagnosed with cancer during their life time; more importantly, 1 out of every 5 people is at risk of dying from cancer. Melanoma is the sixth most common cancer in US, and the number of melanoma cases diagnosed annually is increasing faster than that of any other cancer. Currently, there are five types of standard treatment for metastatic melanoma: surgery, chemotherapy, radiation therapy, immunotherapy (like IL-2, anti-PD1/PD-L1 or anti-CTLA-4) and targeted therapy (e.g., BRAF inhibitor or anti-VEGF). Malignant melanoma can metastasize to all organs of the human body. Autopsy reports from different studies show that multiple organ involvement is a common feature in advanced melanoma, often involving lymph nodes, lungs, livers, bones, kidneys and brain. Melanoma is thought to progress from primary tumor to regional lymph nodes and then to distant metastatic sites. In metastatic cutaneous melanoma, an increased number of circulating tumor cells (CTCs) is associated with poor overall survival. Studies have suggested that certain components of the tissue microenvironment, such as monocytes or macrophages, can facilitate the survival and establishment of metastatic tumors. Therefore, effectively controlling or eliminating establishment of disseminated cancer cells (including CTCs) in vital organs will be a key to increased cancer survival.

### SUMMARY

The present disclosure, in some aspects, provides humanized mouse models (and related methods) useful for, inter alia, studying human cancer metastasis. More particularly, in some embodiments, the present disclosure provides NSG™ (non-obese diabetic (NOD) severe combined immunodeficiency (SCID) gamma) mice that transgenically express human stem cell factor (SCF)/granulocyte-macrophage colony-stimulating factor (GM-CSF)/interleukin-3 (IL-3) (NSG™-SGM3, i.e., NOD.Cg-Prkdc^{scid} Il2rg^{tm1Wjl} Tg(CMV-IL3,CSF2,KITLG)1Eav/MloySzJ) and are engrafted with human CD34⁺ hematopoietic progenitor cells and human metastatic cells (e.g., metastatic melanoma cells). These mice can promote secondary metastatic colonization, modeling the interplay between human tumors and the human immune system. Unexpectedly, not all human melanoma cell lines tested resulted in metastasis. Thus, the humanized mouse models provided herein enable the study of the differences between metastatic and non-metastatic human melanomas dependent on human myeloid cells. These humanized mouse models thus enable mechanistic and pre-clinical studies for the development of novel treatment strategies targeting human-specific molecular pathways controlling cancer (e.g., melanoma) dissemination.

Mouse models have been used extensively to study human diseases in vivo to circumvent the complexity of working with human patients. There are, however, important differences between mouse and human immune system, particularly in the innate immune cells and in mechanisms for sensing tissue damage.

Virtually all cancer-related mortality is due to metastatic disease. To date, most studies of tumor metastasis have been based on syngeneic or genetically engineered mouse models or on human cancer lines (cell line-derived xenograft CDX) and/or patient-derived xenograft (PDX) tumors that are implanted in immunodeficient mice. These experimental systems insufficiently model the interplay between human tumors and the human immune system, which plays a crucial role in the tumor microenvironment and has been shown to significantly impact tumor dissemination and responsiveness to anti-cancer therapies. Alternative approaches and models for assessing cancer metastasis are needed.

The present disclosure addresses the need for a humanized mouse model of metastatic cancer by providing a model system in which the differences between metastatic and non-metastatic pathways can be studied. As indicated above, these mice closely model the interplay between human tumors and the human immune system, only in some cases developing secondary metastases in multiple distant organs.

Thus, the present disclosure provides, in some aspects, methods of producing and/or assessing a transgenic humanized mouse model of metastatic cancer, comprising delivering to a transgenic NSG™-SGM3 mouse human CD34+ hematopoietic progenitor cells to produce a humanized mouse, and delivering to the humanized mouse human cancer cells (e.g. metastatic and/or non-metastatic) to produce a cancer mouse model. In some embodiments, metastasis is assessed, for example, by identifying tumor nodules (comprise metastatic human cancer cells) in organs of the mouse model. In some embodiments, the human cancer cells are human metastatic cancer cells, such as are human metastatic melanoma cells (for example, A375, A2058, and/or Me275 cells). In some embodiments, the human metastatic cancer cells are human metastatic breast cancer cells. In some embodiments, the human metastatic cancer cells are human metastatic pancreatic cancer cells.

In some aspects, the present disclosure provides methods of assessing properties and/or pathways of human cancer cells, the methods comprising delivering to a transgenic humanized NSG™-SGM3 mouse human cancer cells, and assessing one or more properties and/or pathways of the human cancer cells. In some embodiments, the transgenic humanized NSG™-SGM3 mouse comprises human CD34+ hematopoietic progenitor cells.

In some embodiments, the human cancer cells are selected from human melanoma cells, human breast cancer cells, and human pancreatic cancer cells.

In some embodiments, the human cancer cells are metastatic human melanoma cells. In other embodiments, the human cancer cells are non-metastatic human melanoma cells.

In some embodiments, the step of assessing comprises assessing metastasis of the human cancer cells to distant organs of the transgenic humanized NSG™-SGM3 mouse.

In some embodiments, the step of assessing comprises assessing biochemical properties, physiological properties, metabolic processes, signaling pathways, life cycle, and/or chemical composition of the human cancer cells.

In some embodiments, the method further comprises delivering to the transgenic humanized NSG™-SGM3 a therapeutic agent, optionally a monoclonal antibody.

In some embodiments, the method further comprises assessing the effects of the therapeutic agent.

In other aspects, the present disclosure provides methods of producing a transgenic humanized mouse model of metastatic melanoma, comprising delivering to a the transgenic humanized NSG™-SGM3 mouse metastatic human melanoma cells to produce a metastatic melanoma mouse model comprising tumor nodules in organs of the mouse model, wherein the tumor nodules comprise metastatic human melanoma cells. In some embodiments, the transgenic humanized NSG™-SGM3 mouse comprises human CD34⁺ hematopoietic progenitor cells.

Also provided herein, in some aspects are transgenic NSG™-SGM3 mice comprising human CD34+ hematopoietic progenitor cells and human melanoma cells.

In some embodiments, the transgenic mice comprises human CD146+ circulating tumor cells, human CD45+ immune cells, and/or human CD33+ myeloid cells, optionally wherein the CD33+ myeloid cells express cytokine receptors for SCF, cytokine receptors for GM-CSF, and/or cytokine receptors for IL-3.

In some embodiments, the metastatic human melanoma cells are selected from A375, A2058, and Me275 cells.

In some embodiments, 1×10² to 1×10¹⁰ metastatic human melanoma cells are delivered to the transgenic humanized NSG™-SGM3 mouse. For example, 1×10², 1×10³, 1×10⁴, 1×10⁵, 1×10⁶, 1×10⁷, 1×10⁸, 1×10⁹, or 1×10¹⁰ metastatic human melanoma cells may be delivered in the transgenic NSG™-SGM3 mouse.

In some embodiments, the metastatic human melanoma cells are delivered subcutaneously.

In some embodiments, the tumor nodules are present in the mouse liver and/or spleen.

In some embodiments, the level of serum lactate dehydrogenase (LDH) in the metastatic melanoma mouse model is elevated relative to a control transgenic NSG™ mouse that does not comprise the metastatic human melanoma cells.

Other aspects of the present disclosure provide methods of producing a transgenic humanized mouse model of metastatic breast cancer, comprising delivering to a the transgenic humanized NSG™-SGM3 mouse metastatic human breast cancer cells to produce a metastatic breast cancer mouse model comprising tumor nodules in organs of the mouse model, wherein the tumor nodules comprise metastatic human breast cancer cells.

Yet other aspects of the present disclosure provide methods of producing a transgenic humanized mouse model of metastatic pancreatic cancer, comprising delivering to a the transgenic humanized NSG™-SGM3 mouse metastatic human pancreatic cancer cells to produce a metastatic pancreatic cancer mouse model comprising tumor nodules in organs of the mouse model, wherein the tumor nodules comprise metastatic human pancreatic cancer cells.

### BRIEF DESCRIPTION OF DRAWINGS

FIGS. 1A-1H: Humanized NSG™-SGM3 mice promote melanoma metastasis. (FIG. 1A) The construction of humanized mice for human melanoma model, outline of the experiment. Mice were engrafted with CD34+ HPCs at 4 weeks, implanted with melanoma cells subcutaneously (SC) at 14-16 weeks, and analyzed at 22-24 weeks of age (8 weeks post tumor implantation). (FIG. 1B) hNSG™ and hNSG™-SGM3 (hSGM3) mice were implanted with 1×10⁷ Me275 cells SC. The tumor volume was measured weekly. 14 hNSG™ and 15 hNSG™-SGM3 mice from 3 independent experiments with 2 CD34+ HPC donors. (FIG. 1C) Number of macroscopically tumor in the spleen and liver. n=14-15 mice with 2-tailed Mann Whitney test. (FIG. 1D) Serum LDH level measured in hNSG™ or hNSG™-SGM3 mice at 7-8 weeks after 1×10⁷ Me275 SC implantation. n=10 mice with 2-tailed Mann Whitney test. (FIG. 1E) The correlation of serum LDH with the number of visceral metastasis (spleen, liver and kidney). n=10 mice with Spearman test. (FIG. 1F) Mice bearing luciferase-labeled Me275 tumor were harvested and individual organ was analyzed by IVIS. Representative images of 3 hNSG™-SGM3 mice at 8 weeks after 1×10⁶ Me275 SC implantation. aLN, auxiliary lymph node. mesLN, mesenteric lymph node. na, not analyzed. (FIG. 1G) hNSG™ and hNSG™-SGM3 mice were implanted with 1×10⁶ Me275 cells labeled with luciferase. The growth of tumors was monitored weekly by IVIS imaging system (n=2 mice per group). (FIG. 1H) Mice bearing luciferase-labeled Me275 tumor were harvested at different time points and individual organ was analyzed by IVIS imaging system. (FIG. 1I) Macroscopic tumor measured in the liver of hNSG™ or hNSG™-SGM3 reconstituted with different hCD34+ HPC donors and implanted SC with 1×10⁷ or 1×10⁶ Me275 cells for 8 weeks. n=2-13 mice with 2-tailed Mann Whitney test, demonstrating humanized NSG™-SGM3 mice promoter melanoma metastasis.

FIGS. 2A-2E: Melanoma dissemination is associated with circulating tumor cells. (FIG. 2A) hNSG™-SGM3 mice were injected IV with titrated number of Me275 cells labeled with luciferase. Tumor growth were monitored weekly by IVIS. Representative image at day 28. (FIG. 2B) Summary of tumor load measured by total photon emission with IVIS from 2 mice per treatment group. (FIG. 2C) Flow cytometry characterization of CTCs from hNSG™ or hNSG™-SGM3 mice bearing Me275 tumor for 8 weeks. Representative FACS plots on CTCs gated as DAPI−, mCD45−, HLA-ABC+ and CD45− cells with expression of CD44 and CD146. (FIG. 2D) Summary of the frequency of CTC as in FIG. 2C. n=5-10 from two independent experiments with 2-tailed Mann Whitney test. (FIG. 2E) The tumor growth curve in hNSG™-SGM3 mice implanted with 1×10⁴ Me275 cells or sorted CTCs from hNSG™-SGM3 mice bearing Me275 tumor (n=2 mice per group).

FIGS. 3A-3F: Melanoma dissemination is linked with cell cycle status of melanoma cells. (FIG. 3A) The tumor growth curve in hNSG™-SGM3 mice implanted with 1×10⁶ cells from different melanoma cell lines SC (n=3-8 mice per group). (FIG. 3B) Macroscopic tumor in the liver and visceral organs of hNSG™-SGM3 mice implanted SC with 1×10⁶ cells from different melanoma cell lines (n=3-32 mice per group). (FIG. 3C) The frequency of CTC in hNSG™-SGM3 mice bearing different melanoma cell lines in (FIG. 3B). (FIG. 3D) Flow cytometry characterization of proliferation status with nuclei staining of Ki-67 and DNA on Me275 or Colo829 cells from in vitro culture. (FIG. 3E) Summary of (FIG. 3E) from 3 independent experiments. 2way ANOVA with Bonferroni's multiple comparisons test. (FIG. 3F) Flow cytometry characterization of proliferation status with Ki-67 and DAPI staining on CD146+ CTCs from hNSG™-SGM3 mice bearing Me275 tumor or Colo829 tumor for 8 weeks. FACS plot from blood samples pooled from 8-12 mice.

FIGS. 4A-4E: VEGFA contributes to metastatic dissemination. (FIG. 4A) Outline of the experiment. NSG™-SGM3 mice were engrafted with CD34+ HPCs at 4 weeks and implanted with 1×10⁶ luciferase-labeled Me275 cells SC at 16 weeks of age. At day 14 post tumor implantation, mice were treated with PBS or anti-VEGFA IP at 10 mg/kg and 5 mg/kg every 5 days thereafter for a total of 8 treatments. (FIG. 4B) The tumor growth curve in hNSG™-SGM3 mice. Values are mean±s.d. (FIG. 4C) Macroscopically tumor observed in FIG. 4B. n=16-17 mice with 2-tailed Mann Whitney test. (FIG. 4D) Summary of tumor load measured by IVIS imaging system at different organs of hNSG™-SGM3 mice (FIG. 4B). n=11-12 mice with 2way ANOVA test. (FIG. 4E) Quantification of VEGFA+ cells from three hNSG™-SGM3 mice at 7-8 weeks after 1×10⁷ Me275 SC implantation. n=3 mice with 2-tailed paired t test.

FIGS. 5A-5E: Melanoma dissemination in hNSG™-SGM3 mice is dependent on the human CD33+ cells. (FIG. 5A) The tumor growth curve comparing NSG™-SGM3 and hNSG™-SGM3 mice implanted with 1×10⁷ Me275 cells SC. (FIG. 5B) Macroscopic tumor observed in (FIG. 5A). n=12-13 mice with 2-tailed Mann Whitney test. (FIG. 5D) Outline of the experiment. (FIG. 5D) The tumor growth curve in NSG™-SGM3 mice after adoptive transfer of 1×10⁷ hCD33+ or hCD33− cells IV and subsequently implanted with 1×10⁷ Me275 cells SC. (FIG. 5E) Macroscopic tumor in the liver, spleen and visceral organs of NSG™-SGM3 mice after adoptive transfer of hCD45, hCD33+ or hCD33− cells. n=7-9 mice with one-way ANOVA test.

FIGS. 6A-C: Pro-metastatic human CD33+ cells in hNSG™-SGM3 mice resemble CD33+ infiltrate in human melanoma tumor. (FIG. 6A) Venn diagram illustrates the human myeloid genes expressed in patient melanoma tumor and CD33+ cells from hNSG™-SGM3 mice. (FIG. 6B) Ingenuity pathway analysis on the common myeloid genes for the upstream regulator. Top 5 regulators, STAT3, RELA, STAT1, NFKB1, NR3C1 were illustrated with top 25 genes of each network. (FIG. 6C) Heatmap illustrates the transcriptional expression of metastasis-associated genes in 533 common myeloid genes.

FIGS. 7A-7F: Humanized NSG™-SGM3 promote breast cancer metastasis. (FIG. 7A) The construction of humanized mice for human breast cancer model, outline of the experiment. Mice were engrafted with CD34+ HPCs at 4 weeks, implanted with breast cancer cells at 12-16 weeks, and analyzed at 16-24 weeks. (FIG. 7B) hNSG™ and hNSG™-SGM3 mice were implanted with 1×10⁷ MDA-MB231 cells. The tumor volume was measured every week. (FIG. 7B) Macroscopic tumor nodules measured in the liver of hNSG™ or hNSG™-SGM3 at 5 weeks after 1×10⁷ MDA-MB231 SC implantation. (FIG. 7C) Macroscopically tumor nodules measured in the liver of hNSG™ or hNSG™-SGM3 at 5 weeks after 1×107 MDA-MB231 SC implantation. (FIG. 7D) Outline of the experiment with human breast cancer model in SGM3 mice with tumor resection. (FIG. 7E) hNSG™ and hNSG™-SGM3 mice were implanted with 3×10⁶ Hs578t and HCC1806 breast cancer cells. The tumor volume was measured every week. (FIG. 7F) Macroscopic tumor nodules measured in the liver and lung of hNSG™ or hNSG™-SGM3 with or without tumor resection.

FIGS. 8A-8C: Humanized NSG™-SGM3 promote pancreatic cancer metastasis. (FIG. 8A) The construction of humanized mice for human pancreatic cancer model, outline of the experiment. Mice were engrafted with CD34+ HPCs at 4 weeks, implanted with pancreatic cancer cells at 12-16 weeks, and analyzed at 22-26 weeks. (FIG. 8B) hNSG™ and hNSG™-SGM3 mice were implanted with 1×10⁶ MiaPaCa2 and Panel cells. The tumor volume was measured every week. (FIG. 8C) Macroscopic tumor nodules measured in the liver, spleen and all distant organs of hNSG™ or hNSG™-SGM3 at 10 weeks after 1×10⁶ MiaPaCa2 and Panel cells SC implantation.

### DETAILED DESCRIPTION

Metastatic melanoma remains an incurable disease for some patients due to treatment resistance and metastatic dissemination. Current experimental systems insufficiently model the interface between human melanoma and human immune cells. The data provided herein show that NSG™ mice with transgenic expression of human hematopoietic cytokines SCF/GM-CSF/IL-3 (NSG™-SGM3) when engrafted with human CD34+ hematopoietic progenitor cells, and implanted with Me275 human melanoma cell line subcutaneously, developed multi-organ distant melanoma tumors. This was linked with the presence of circulating tumor cells and elevated serum biomarker lactate dehydrogenase (LDH). Among six melanoma cell lines analyzed, potential to form distant tumors was linked with G0/G1 cell cycle status and proliferative capacity. Treatment with VEGF inhibitor bevacizumab significantly decreased the number of melanoma tumors in the spleen but not in the liver. Adoptive transfer experiments revealed the critical role of human CD33+ myeloid cells. Transcriptional profiles of these pro-metastatic human CD33+ myeloid cells were driven by STAT3 and could be detected in melanoma tumor samples from patients. Thus, this model enables mechanistic and pre-clinical studies for the development of treatment strategies targeting human-specific molecular pathways controlling melanoma dissemination.

The treatment landscape of advanced melanoma has changed dramatically over the past decade¹. Improved survival has been documented for patients treated with a blocking antibody targeting the cytotoxic T-lymphocyte-associated protein CTLA-4² alone or combined with therapies targeting another T cell checkpoint, programmed death PD-1 or programmed death ligand PDL-1². However, a significant fraction of patients does not achieve prolonged survival even in combination therapy trials³. Furthermore, targeted therapy in BRAF-mutant advanced melanoma, alone or combined with MEK inhibitors, is linked with acquired resistance and extended survival in less than 50% of patients⁴. Thus, additional therapies are needed to improve outcomes for patients with advanced melanoma.

The main clinical challenge is the metastatic spread of treatment-resistant melanoma.

Indeed, melanoma can metastasize to all organs of the human body. Based on the autopsy reports from different studies, multiple organ involvement is common and often involves lymph nodes, lungs, livers, bones, kidneys and brain^{5, 6, 7, 8, 9}. Among distant metastasis, patients with non-pulmonary visceral metastasis have the worst prognosis¹⁰. The higher number of circulating tumor cells (CTCs) in metastatic cutaneous melanoma is associated with poor overall survival¹¹.

The interplay between human tumor and the human immune system has been shown to significantly impact tumor dissemination and responsiveness to anti-cancer therapies. Mouse models of human cancer have been used extensively to study cancer in vivo to circumvent the complexity of studies in human patients and enable the identification of causative links. Although used for proof-of-concept studies and demonstration of genetic links, murine models often inadequately recapitulate the human cancer-immune interface. Furthermore, there are important differences between mouse and human immune systems¹², particularly in the innate immune cells and in mechanisms for sensing tissue damage^{12, 13}. For example, C-type lectins are innate receptors expressed on antigen-presenting cells that are involved in the recognition of glycosylated pathogens and self-glycoproteins^{14, 15}. Dendritic cell immunoreceptor (DCIR), the only classical C-type lectin that contains an intracellular immunoreceptor tyrosine-based inhibitory motif (ITIM), exists in four isoforms in the mouse while only one has been identified in the human¹⁶. Similarly, NOD-like innate immune sensors (NLR's) differ significantly with mice having around 10 more genes than the approximately 20 found in humans¹⁷. Major differences also exist between human and mouse NK cells¹². Thus, the knowledge gained from mouse studies frequently cannot be extrapolated to humans, as these differences could deeply impact the regulation of tissue homeostasis and the underlying inflammation and recognition of tissue antigens.

In an effort to resolve this need, the present disclosure utilizes a transgenic humanized mouse model, which is an immunodeficient mouse with a transplanted human immune system^{18, 19, 20, 21, 22, 23, 24, 25, 26}. The engraftment of immunodeficient mice bearing the gc mutation including NOD-SCID-IL2 gc^{-/-} (NSG) results in multi-lineage human cells development including human B and T cells^{27, 28}. A variant of these immunodeficient mice, used herein, is based on NSG™ mice with transgenic expression of human Stem Cell Factor (SCF), Granulocyte Macrophage-Colony Stimulating Factor (GM-CSF) and Interleukin (IL)-3 (NSG™-SGM3)^{31, 32}. Initial studies demonstrated that, when transplanted with hCD34+ HPCs, these mice efficiently support the development of human immune cells, especially the CD33+ myeloid cells as well as CD4+Foxp3+ regulatory T cells, as compared to non-transgenic counterparts³³.

The present disclosure shows that humanized NSG™-SGM3 (hNSG™-SGM3) mice develop distant metastasis involving multiple organs when implanted with human melanoma cell lines subcutaneously. This model enables the pre-clinical screening of novel immunotherapy, especially those that aim to block metastasis.

As used herein, the term “NSG™” refers to the humanized mouse model of NOD scid gamma (NOD.Cg-Prkdcscid Il2rgtm1Wjl/SzJ mice). The mice carry two mutations on the NOD/ShiLtJ genetic background: severe combined immune deficiency (scid) and a complete null allele of the IL2 receptor common gamma chain (IL2rgnull). These mice are immunodeficient. The terms “NSG™” and “humanized NSG™” (hNSG™) are used herein interchangeably.

As used herein, the term “NSG™-SGM3” refers to NSG™ mice that are triple transgenic mice expressing human IL3, human GM-CSF and human SCF as the combined features of (in the background of) the NSG™ mouse with cytokines that support the stable engraftment of myeloid lineages and regulatory T cell populations. The terms “NSG™-SGM3” and “humanized NSG™-SGM3” (hNSG™-SGM3) are used herein interchangeably.

Provided herein, in some aspects, are transgenic humanized mouse models of human metastatic cancer and methods of producing the transgenic humanized mouse models.

Thus, in some aspects, the present disclosure provides methods of producing transgenic humanized mouse models of human cancer, such as human metastatic cancer. In some embodiments, provided herein are methods of producing transgenic humanized mouse models of human metastatic melanoma. In other embodiments, provided herein are methods of producing transgenic humanized mouse models of human metastatic breast cancer. In yet other embodiments, provided herein are methods of producing transgenic humanized mouse models of human metastatic pancreatic cancer.

The methods, in some embodiments, include delivering to a transgenic NSG™-SGM3 mouse human CD34⁺ hematopoietic progenitor cells to produce a humanized mouse. As discussed above, the NSG™-SGM3 mouse, a variant of the NOD-SCID-IL2 gc^{-/-} (NSG) mouse, express human SCF, GM-CSF, and IL-3.

Herein, cells and/or tissue may be delivered to a mouse by any means known in the art. The process of delivering a cell and/or tissue may be referred to as “engraftment” or “implantation.” Examples of delivery routes include, but are not limited to, intravenous injection, subcutaneous injection (e.g., into the flanks of the mice), intramuscular injection, intraocular injection, intraventricular injection, intrathecal injection, intrafemural injection, and surgical implantation. In some embodiments, cells are delivered to a mouse by intravenous injection into the tail vein.

In some embodiments, human CD34⁺ hematopoietic progenitor cells (HPCs) (e.g., from fetal liver, full-term cord blood, or adult bone marrow or blood) are delivered to a mouse (e.g., a transgenic NSG™-SGM3 mouse). Such a mouse is considered to be “engrafted with” human CD34⁺ HPCs. In some embodiments, a mouse is engrafted with 1×10³ to 1×10⁸ CD34⁺ HPCs. For example, a mouse may be engrafted with 1×10³ to 1×10, 1×10³ to 1×10⁷, 1×10³ to 1×10⁶, 1×10³ to 1×10⁵, 1×10³ to 1×10⁴, 1×10⁴ to 1×10, 1×10⁴ to 1×10⁷, 1×10⁴ to 1×10⁶, or 1×10⁴ to 1×10⁵ CD34⁺ HPCs. In some embodiments, a mouse is engrafted with 1×10³, 1×10⁴, 1×10⁵, 1×10⁶, 1×10⁷, or 1×10 CD34⁺ HPCs. Mice (e.g., NSG™-SGM3 mice) engrafted with CD34⁺ HPCs may be referred to herein as “humanized mice.”

In some embodiments, human cancer cells are delivered to a mouse (e.g., a humanized mouse). Examples of human cancer cells include, but are not limited to, melanoma cells, breast cancer cells, pancreatic cancer cells, lung cancer cells, leukemia cells, lymphoma cells, renal cancer cells, liver cancer cells, bone cancer cells, adrenal cancer cells, colon cancer cells, ovarian cancer cells, bladder cancer cells, and cervical cancer cells.

In some embodiments, a mouse (e.g., a humanized mouse) is engrafted with 1×10³ to 1×10⁸ human cancer cells. For example, a mouse may be engrafted with 1×10³ to 1×10⁸, 1×10³ to 1×10⁷, 1×10³ to 1×10⁶, 1×10³ to 1×10⁵, 1×10³ to 1×10⁴, 1×10⁴ to 1×10⁸, 1×10⁴ to 1×10⁷, 1×10⁴ to 1×10⁶, or 1×10⁴ to 1×10⁵ human cancer cells. In some embodiments, a mouse is engrafted with 1×10³, 1×10⁴, 1×10⁵, 1×10⁶, 1×10⁷, or 1×10⁸ human cancer cells.

In some embodiments, the human cancer cells are human melanoma cells. Examples of human melanoma cell lines that may be used as provided herein include Me275 (LAU-Me275 (RRID:CVCL_S597)), which were established from surgically excised melanoma metastases from patients LAU50 and Me290 (RRID:CVCL_S598), A2058 (ATCC® CRL-11147™), A375 (ATCC® CRL-1619™), Colo-829 (ATCC® CRL-1974™), SKmel5 (ATCC® HTB-70™), SKmel24 (ATCC® HTB-71™), MDA-MB231 (ATCC® CRM-HTB-26™), Hs578t (ATCC® HTB-126™), HCC1806 (ATCC® CRL-2335™), MiaPaCa-2 (ATCC® CRL-1420™), and Panel (ATCC® CRL-1469™), WM852 (Rockland Immunochemicals), and WM1366 (Rockland Immunochemicals). Other melanoma cell lines may be used.

In some embodiments, a transgenic NSG™-SGM3 mouse comprises human CD34+ hematopoietic progenitor cells and metastatic human A375 cells. In some embodiments, a transgenic NSG™-SGM3 mouse comprises human CD34+ hematopoietic progenitor cells and metastatic human A2058 cells. In some embodiments, a transgenic NSG™-SGM3 mouse comprises human CD34+ hematopoietic progenitor cells and metastatic human Me275 cells.

In some aspects, the present disclosure provides methods of assessing metastasis of a human cancer cell in a humanized mouse. Metastasis, as used herein, refers to the development of secondary malignant growths at a distance from the primary site of a cancer. The primary site refers to the initial location where the cancer development. The sites of secondary metastases can be predicted in human cancers. The most common sites of melanoma metastasis are the liver, lungs, bones, and brain; the most common sites of breast cancer metastasis are bone, lungs, lymph nodes, liver, and brain; and the most common sites of pancreatic cancer metastases are the liver, lungs, bone, brain, and stomach. The secondary site can be, but is not limited to: lymph nodes, lung, brain, pancreas, liver, spleen, bone, bone marrow, kidney, or skin.

In some aspects, the present disclosure provides a therapy and/or agent to halt the metastasis of cancer in a transgenic mouse. Agents which halt the metastasis of cancer are well-known in the art and include, for example, monoclonal antibodies, such as anti-PD-1 antibodies and anti-CTLA-4 antibodies. Other monoclonal antibodies that may be used here include, for example, trastuzumab, ipilimumab, pembrolizumab, catumaxomab, and bevacizumab. Other therapies may be administered, in some embodiments, such as radiation therapy and/or chemotherapy, using latrunculin A, chondramide, and/or TR-100, for example.

In some embodiments, delivering human cancer (e.g., melanoma) cells to a humanized mouse metastatic produces a metastatic cancer (e.g., melanoma) mouse model comprising tumors in organs of the mouse model. These tumors, in some embodiments, comprise metastatic human cancer (e.g., melanoma) cells. These tumors may form in lymph nodes, lung, brain, pancreas, liver, spleen, bone, bone marrow, kidney, and/or skin.

In some embodiments, the level of serum lactate dehydrogenase (LDH) in the mouse models is elevated relative to a control transgenic NSG™ mouse that does not comprise the metastatic human melanoma cells. For example, the level of LDH may be elevated by at least 2-fold, at least 3-fold at least 4-fold, at least 5-fold, at least 6-fold, at least 7-fold, at least 8-fold, at least 9-fold, or at least 10-fold. In some embodiments, the level of serum lactate dehydrogenase (LDH) in the mouse models is elevated by 2-fold to 10-fold.

In some embodiments, the mouse models comprise human CD146+ circulating tumor cells, human CD45+ immune cells, and/or human CD33+ myeloid cells (e.g., at least 10%, 20%, 30%, 40%, or 50% more cells relative to a control NSG™ mouse that does not comprise metastatic human melanoma cells).

In some embodiments, the mouse models comprise human CD33+ myeloid cells that express cytokine receptors for SCF, cytokine receptors for GM-CSF, and/or cytokine receptors for IL-3.

The following examples are provided to further illustrate various non-limiting embodiments and techniques of the present methods, including experiments performed in developing the present methods. It should be understood, however, that these examples are meant to be illustrative and do not limit the scope of the claims. As would be apparent to skilled artisans, many variations and modifications are intended to be encompassed within the spirit and scope of the disclosure.

### EXAMPLES

### Example 1. Humanized NSG™-SGM3 Mice Promote Melanoma Metastasis

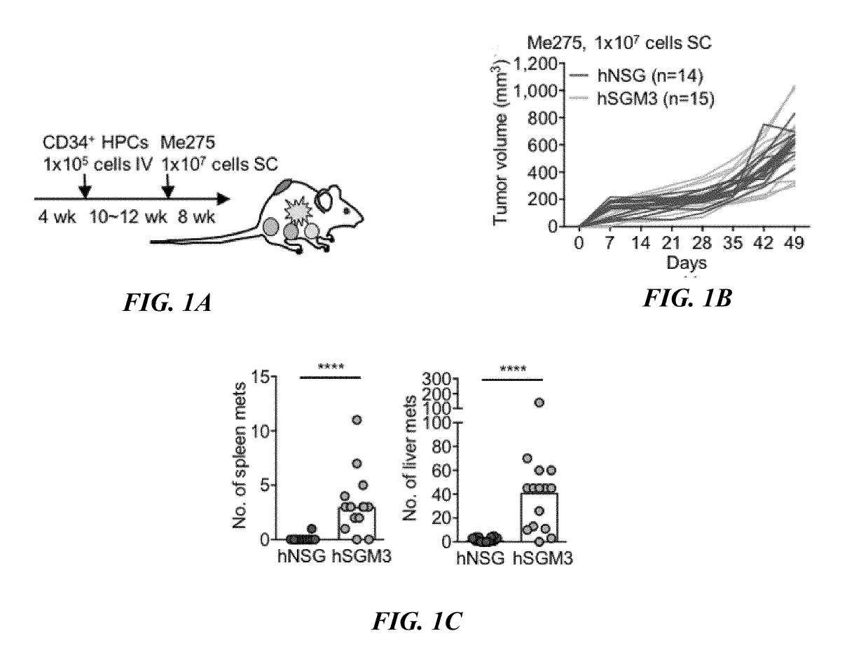

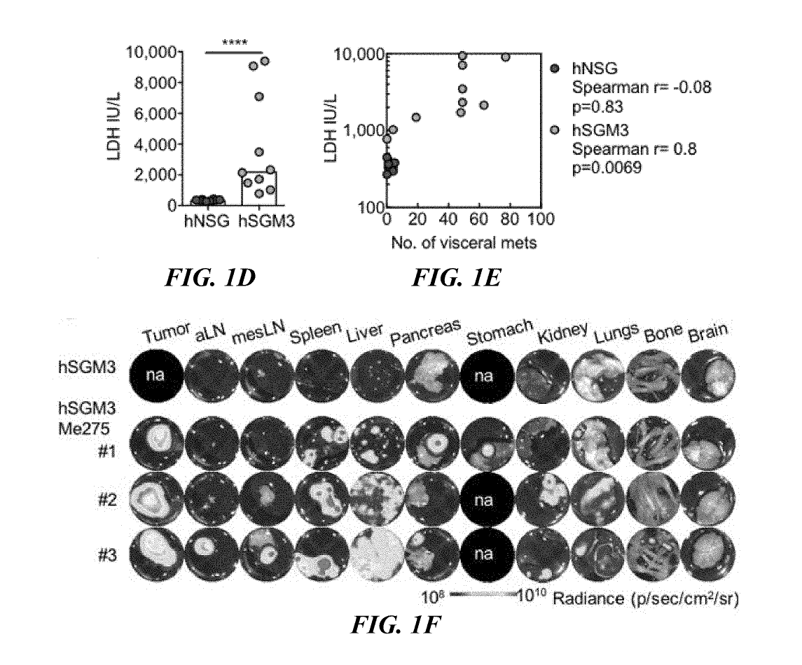

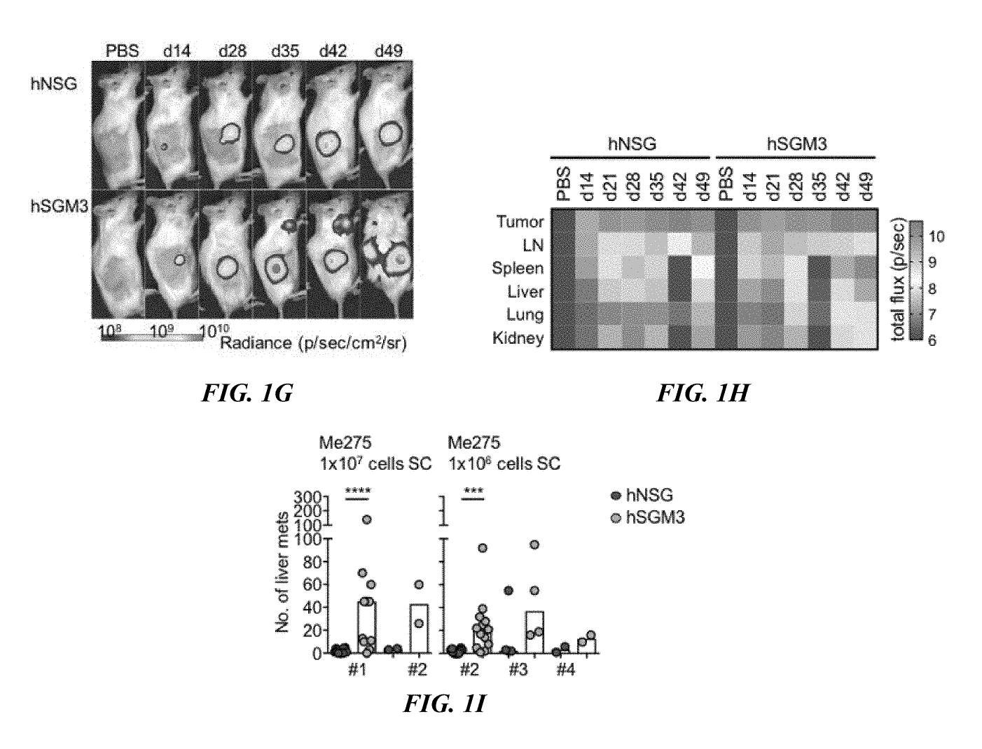

To determine if NSG™-SGM3 mice are suitable to study human malignant melanoma, we first engrafted irradiated NSG™ and NSG™-SGM3 mice with hCD34+ HPCs and 10-12 weeks later implanted 1×10⁷ Me275 human melanoma cells subcutaneously (SC, FIG. 1A and Table 1).

TABLE 1
List of melanoma tumor cells.

| Name | Site | Stage | Melanoma marker |
|---|---|---|---|
| A375 | Skin | MET | CD146 |
| A2058 | Metastatic lymph node | MET | CD146, MART-1, gp100 |
| Colo829 | Skin biopsy | MET | CD146, MART-1, gp100 |
| Me275 | Metastatic lymph node | MET | CD146, MART-1, gp100 |
| Me290 | Metastatic lymph node | MET | CD146, MART-1, gp100 |
| SKmel5 | Metastatic axillary node | MET | CD146, MART-1, gp100 |
| SKmel24 | Metastatic lymph node | MET | CD146, MART-1, gp100 |
| WM852 | Skin, abdomen | MET | CD146 |
| WM1366 | Skin, forearm | VGP | CD146 |

Eight weeks later both strains showed tumor development at implantation site (“primary tumor”) (FIG. 1B). However, on necropsy, hNSG™-SGM3 mice, but not hNSG™ mice, displayed disseminated macroscopic tumors in spleen and liver (FIG. 1C). Immunofluorescence staining of frozen tissue sections from involved organs revealed expression of melanoma antigens MART-1 and gp100 thus confirming the presence of disseminated melanoma cells in hNSG™-SGM3 mice (data not shown). In patients with metastatic melanoma, elevated serum lactate dehydrogenase (LDH) is a strong independent prognostic factor correlated with survivall^{10, 34}. Accordingly, hNSG™-SGM3 mice bearing melanoma displayed elevated serum LDH as compared with hNSG™ mice (FIG. 1D), which was correlated with the total number of visceral (spleen, liver and kidney) metastasis (FIG. 1E). To further determine the extent of melanoma dissemination in hNSG™-SGM3 mice, we analyzed organ involvement using in vivo imaging system (IVIS) to detect luciferase-labeled Me275 melanoma cells. Tumors were imaged in vivo or ex vivo immediately upon necropsy (FIG. 1F, 1G). This analysis revealed luciferase signal in multiple organs including both lymphoid (LNs and spleen) and non-lymphoid (liver, pancreas, stomach, kidney, lungs and bone) organs (FIG. 1F). Thus, metastatic spread of melanoma in hNSG™-SGM3 is systemic and resembles disease pattern observed in melanoma patients^{5, 6, 7, 8, 9}. This pattern of disease dissemination was independent of the number of implanted melanoma cells (range 1×10⁶ to 1×10⁷) and/or of the donor of hCD34+ HPCs used to engraft the mice (n=4 donors tested) (FIG. 1I). Kinetic experiments following the appearance of macroscopic tumors as well as luminescence signal in vivo revealed that Me275 cells require at least 28-35 days for LN tumors and 42 days for visceral tumors to appear (FIG. 1G, 1H). Thus, hNSG™-SGM3 mice support the metastatic spread of human Me275 melanoma cells.

### Example 2. Melanoma Dissemination is Associated with the Presence of Circulating Tumor Cells

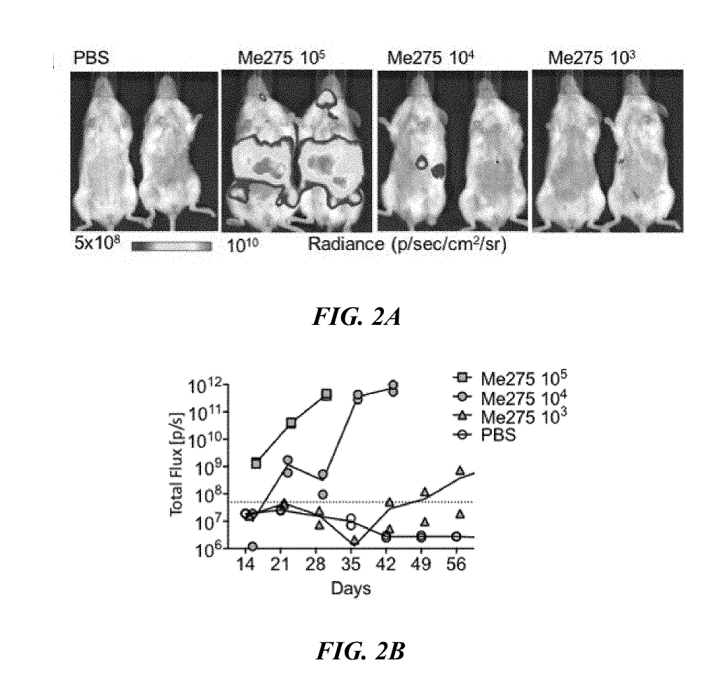

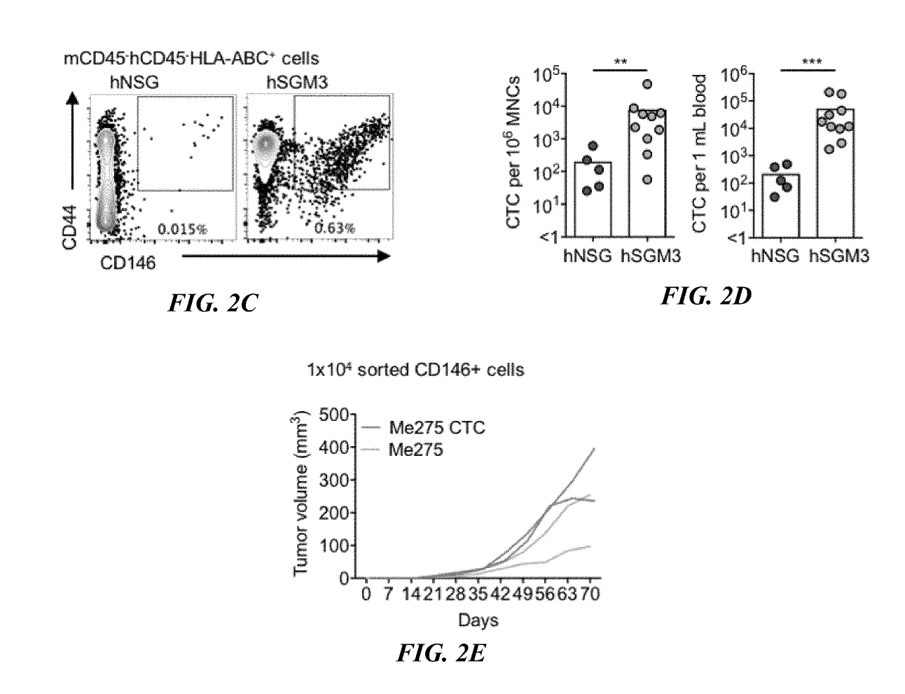

To determine if circulating melanoma cells can lead to disease dissemination in hNSG™-SGM3 mice, we injected titrated number (10³-10⁵) of Me275 cells intravenously (IV) and followed the disease progression by IVIS in vivo over time. hNSG™-SGM3 mice showed tumor development in several organs including spleen and liver (FIG. 2A, 2B). These results prompted us to analyze whether development of systemic tumors is associated with the presence of CTCs in hNSG™-SGM3 mice implanted SC with Me275 melanoma cells. To this end, we first established and verified the panel of surface markers that could be used to detect CTCs (data not shown) and used non-specific markers (HLA-ABC and CD44) and melanoma-specific marker (CD146) to identify melanoma cells in the blood (data not shown). As shown in FIGS. 2C and 2D hNSG™-SGM3 mice bearing Me275 SC tumors showed significantly higher frequency of CD146+ CTCs than hNSG™ mice. When sorted from the blood of hNSG™-SGM3 mice, CD146+ CTCs displayed expression of MART-1 and gp100 confirming that they are melanoma cells (data not shown). Furthermore, when implanted SC in tumor-naïve hNSG™-SGM3 mice, the CD146+ CTCs established subcutaneous tumors (FIG. 2E) and metastasized to the distant organs (data not shown) comparably to parental Me275 cells. Thus, circulating Me275 melanoma cells in hNSG™-SGM3 mice implanted SC, are viable and can generate tumors in vivo.

### Example 3. Melanoma Dissemination is Linked with Cell Cycle Status of Melanoma Cells

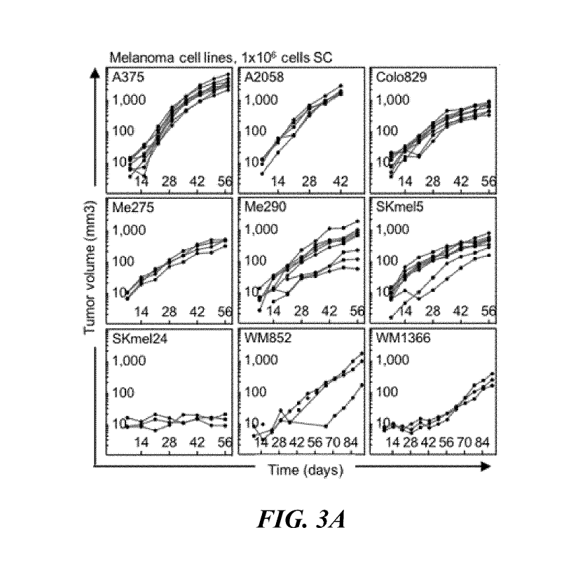

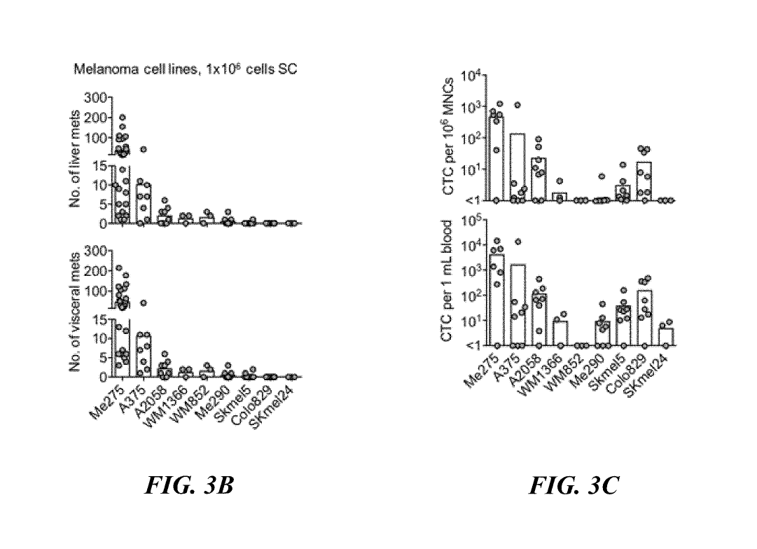

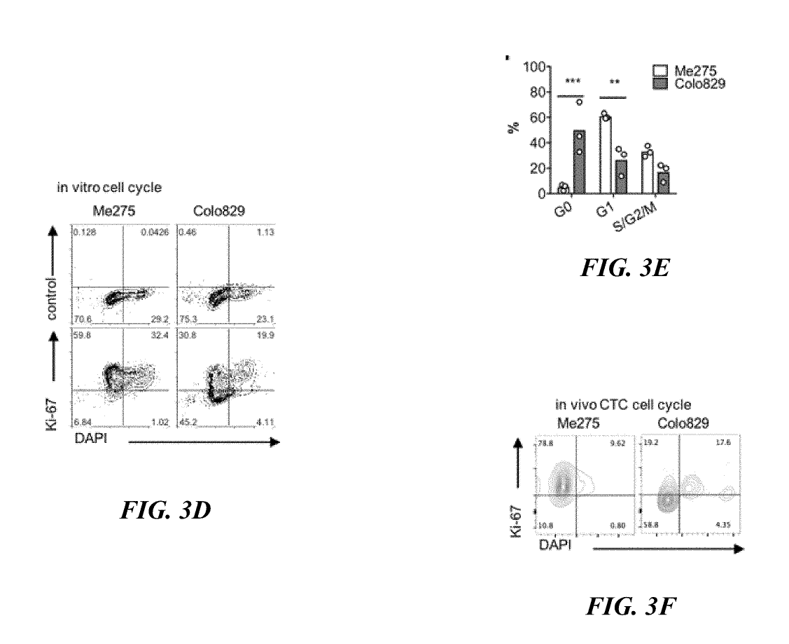

To determine the broader applicability of hNSG™-SGM3 mice to study melanoma dissemination, we tested additional eight different melanoma cell lines, including A375, A2058, Colo829, Me290, SKmel5, SKmel24, WM852, and WM1366 cells (Table 1). All but SKmel24 cells developed primary tumor on SC implantation (FIG. 3A). Further analysis revealed however two types of melanoma cell lines: those that do form distant tumors in hNSG™-SGM3 mice (Me275, A375, A2058 and WM1366) and those that do not (Colo829, Me290, SKmel5, WM852) (FIG. 3B and data not shown). Interestingly, despite relatively high frequency of CTCs in Colo829 cells, no distant tumors could be detected (FIG. 3B, 3C and data not shown). Thus, the presence of circulating melanoma cells is not sufficient to establish tumors in distant organs in hNSG™-SGM3 mice.

Kinetic experiments showed above revealed that Me275 cells require time to form tumors in distant organs (FIGS. 1A-1H). This suggested that melanoma cells that seed the organs need to proliferate locally to establish macroscopically visible tumor. Accordingly, immunofluorescence analysis of frozen tissue sections of involved organs showed the presence of Ki-67 expressing melanoma cells (data not shown). Thus, we reasoned that the cell cycle status and proliferative capacity of melanoma cells might be a contributing factor determining whether or not metastatic dissemination will occur. To this end, we analyzed cell cycle status in both Me275 metastatic and Colo829 non-metastatic melanoma cell lines that both exhibit comparatively high frequency of CTCs in hNSG™-SGM3 mice as shown in FIGS. 3B and 3C. Using Ki-67 and DNA staining of cell lines cultured in vitro, we found a significantly higher percentage of Ki-67+ cells in G1 phase among Me275 cells than among Colo829 cells (61±2.2 and 26±11, respectively, p=0.0039) and a significantly higher percentage of Ki-67-cells in G0 phase among Colo829 cells (5.0±2.4 and 50±20, respectively, p=0.0004) (FIG. 3D, 3E). We next carried out cell cycle analysis of CTCs in vivo and found the same pattern of Ki-67 expression and DNA staining with prevalence of cells in G1/G2-M phase among circulating Me275 melanoma cells and a prevalence of non-cycling cells in G0 phase among circulating Colo829 melanoma cells (FIG. 3F). Thus, the capacity of melanoma cells implanted SC in hNSG™-SGM3 mice to proliferate in vivo in distant organs represents a contributing factor to metastatic dissemination.

### Example 4. VEGFA Contributes to Metastatic Dissemination

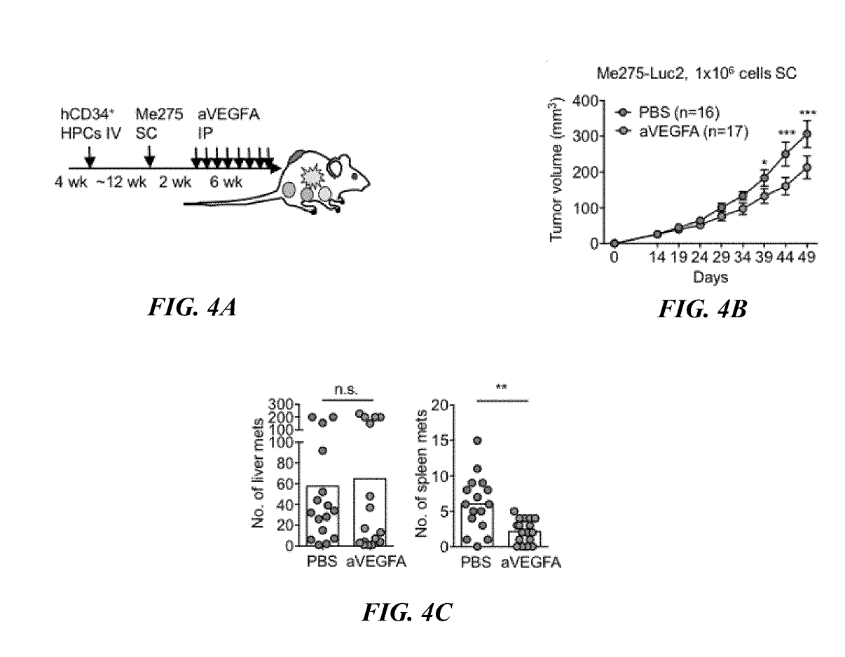

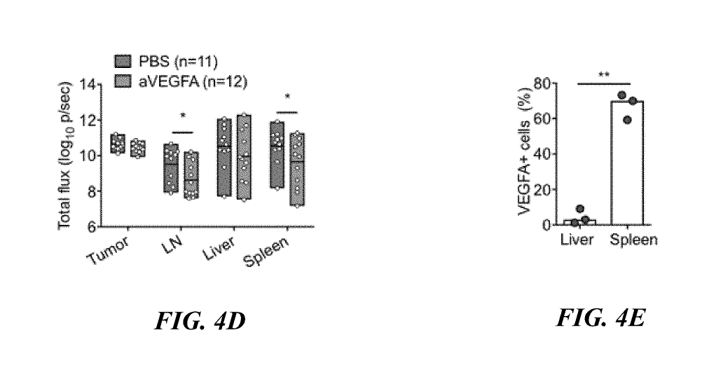

Our results thus far suggested that the metastatic potential of melanoma cell lines in hNSG™-SGM3 mice is linked with the cell cycle status suggesting the need to establish microenvironment supporting cell proliferation. This prompted us to treat the mice with VEGFA inhibitor Avastin as our earlier studies demonstrated that Me275 melanoma cells forming “primary tumors” in humanized mice are responsive to VEGFA¹⁸. To this end, hNSG™-SGM3 mice were implanted with 1×10⁶ Me275 cells SC and at day 14 post-tumor implant they received treatment of Avastin IP at 10 mg/kg and 5 mg/kg every 5 days thereafter for a total of eight times over 40 days (FIG. 4A). Mice were randomly assigned to treatment arms (Avastin vs. saline control), primary tumors were measured in blinded fashion every 5 days and metastatic dissemination was assessed upon necropsy at week 8. In four independent experiments with hCD34+ HPCs from 2 different donors, Avastin treatment impacted the development of primary tumor (306±150 vs. 213±133 on day 49, p=0.0004). (FIG. 4B). Nevertheless, upon necropsy, no difference in the number of macroscopic tumors in the liver was observed (FIG. 4C). However, in three out of four experiments we found a significantly lower number of melanoma tumors in the spleen in mice treated with Avastin (6.1±3.9 vs. 2.2±1.7, p=0.0012) (FIG. 4C and data now shown). The inhibition of metastatic tumors in the spleen was further confirmed in the in vivo imaging experiments where in Avastin-treated mice the luminescence signal was significantly lower when compared to control group (10.55±0.97 vs. 9.65±1.39, p=0.0389) (FIG. 4D). To understand why Avastin controlled the metastasis in the spleen but not in the liver, we analyzed human VEGFA expression in the liver and spleen of hNSG™-SGM3 mice bearing melanoma. We found expression of human VEGFA protein in human CD45+ cells in the spleen but no VEGFA expression in the liver (FIG. 4E and data not shown). Because Avastin can neutralize human but not mouse VEGF³⁵, these results suggest that spleen metastasis were at least partially mediated by human VEGFA produced by human CD45 (hCD45)+ leukocytes.

### Example 5. Melanoma Dissemination in hNSG™-SGM3 Mice is Dependent on the Human CD33+ Cells

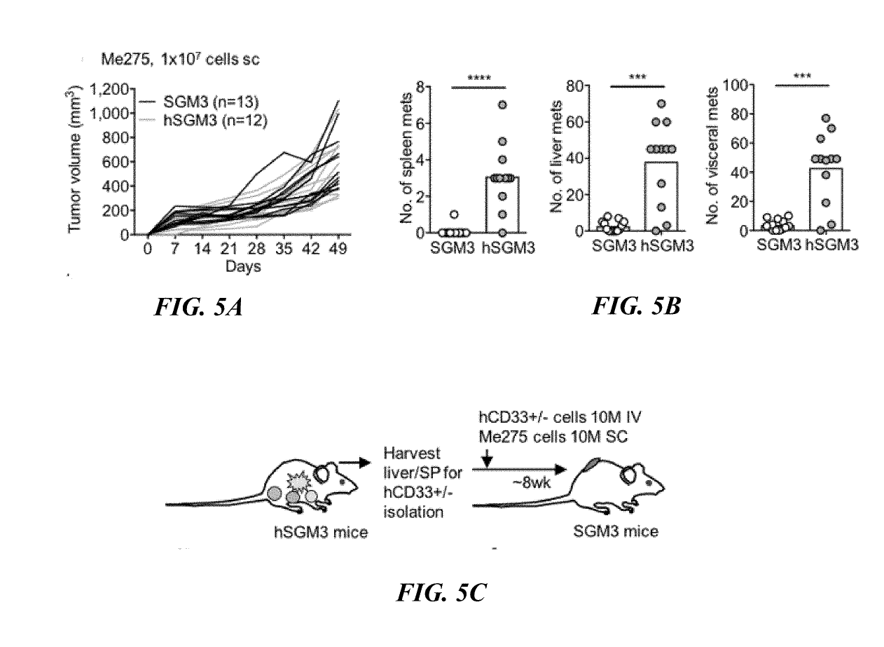

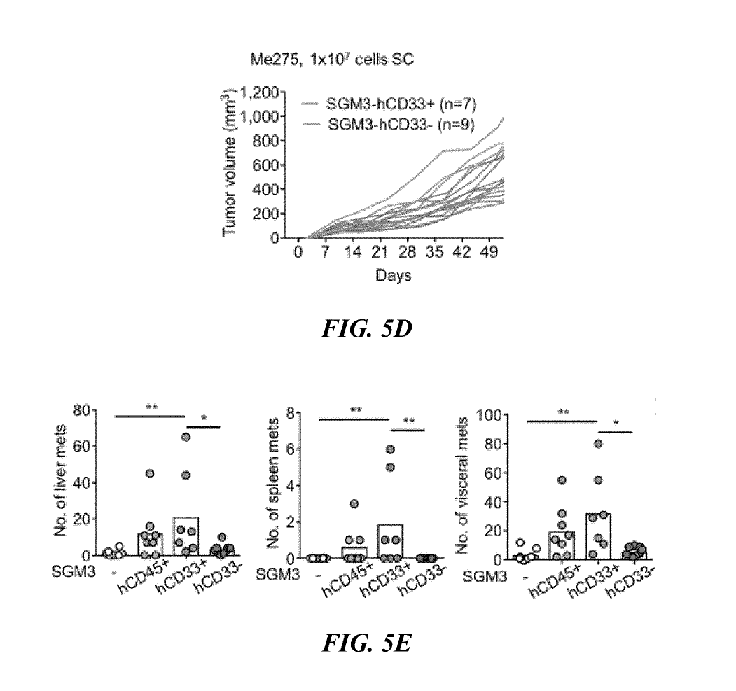

To further investigate the role of hCD45+ cells in formation of tumors in distant organs, we implanted Me275 cells into non-humanized NSG™-SGM3 mice (NSG™-SGM3). Me275 tumor growth at the implantation site was similar to that observed in hNSG™-SGM3 mice (FIG. 5A) suggesting that human cells were not critical for the formation of primary tumor. However, there was no evidence of metastatic dissemination as determined by the lack of macroscopic tumors in visceral organs on necropsy and no detectable MART-1/gp-100 staining in tissues (FIG. 5B and data not shown). We next purified hCD45+ cells from the liver and spleen of tumor-naïve hNSG™-SGM3 mice and adoptively transferred them (1×10⁷) into non-irradiated and tumor-naïve non-humanized NSG™-SGM3 mice and NSG™ mice to generate hCD45+NSG™-SGM3 mice and hCD45+NSG™ mice, respectively. Both strains were subsequently implanted with Me275 cells SC (data not shown). As expected, non-humanized control NSG™-SGM3 mice did not develop metastasis (data not shown). However, adoptive transfer of hCD45+ cells rescued the metastatic dissemination in hCD45+NSG™-SGM3 mice but not in hCD45+NSG™ mice (data not shown). Thus, metastatic dissemination of melanoma in hNSG™-SGM3 mice is driven by human leukocytes and is dependent on host human cytokines SCF, GM-CSF, and IL-3.

When analyzing the composition of hCD45+ cells, we observed enrichment of human CD33+ (hCD33+) cells in peripheral tissues of hNSG™-SGM3 mice as compared to hNSG™ mice (data not shown). To test if hCD33+ myeloid cells account for the support of metastatic dissemination in hNSG™-SGM3 mice, we performed another adoptive cell transfer experiment (FIG. 5C). To this end, hCD45+ cells isolated from the liver and spleen of hNSG™-SGM3 mice, were separated into either CD33+ or CD33− cell fraction by magnetic bead isolation (data not shown), and adoptively transferred into non-humanized NSG™-SGM3 mice to generate hCD33+NSG™-SGM3 mice and hCD33-NSG™-SGM3 mice that were subsequently implanted SC with Me275 cells (FIG. 5D). As shown in FIG. 5E, hCD33+NSG™-SGM3 mice but not hCD33-NSG™-SGM3 mice showed macroscopic tumors in distant organs. Thus, human CD33+ myeloid cells mediate metastatic dissemination of melanoma in hNSG™-SGM3 mice. Furthermore, tissue analysis showed the close proximity of hCD33+ cells to melanoma cells in the metastatic liver and spleen of hNSG™-SGM3 mice, in a manner similar to that of human melanoma tumor sample (data now shown).

### Example 6. Human CD33+ Myeloid Cells in hNSG™-SGM3 Mice Resemble CD33+ Infiltrate in Human Melanoma Tumors

To determine whether hCD33+ cells with pro-metastatic activity in NSG™-SGM3 mice resemble myeloid infiltrate in human metastatic melanoma tumor samples from patients, we analyzed transcriptional profiles with RNAseq of hCD33+ cells purified from the bone marrow, spleen and liver of hNSG™-SGM3 mice at 12-week post hCD34+ HPC transplant as well as of whole tumor sections from 14 patient samples (Table 2).

TABLE 2
List of melanoma patient tumors.

| Tumor ID | Age | Gender | Race | Stage | Site | Treatments |
|---|---|---|---|---|---|---|
| 52067T001 | 87 | F | W | MET | Metastatic axillary node | No |
| 59818T003 | 60 | M | U* | MET | Metastatic jejunum | Radiation |
| 63362T001 | 79 | M | W | MET | Lung | No |
| 63804T005 | 65 | M | W | MET | Metastatic axillary node | No |
| 67135T002 | 57 | M | W | MET | Metastatic axillary node | Radiation |
| 68352T005; | 50 | M | W | MET | Metastatic axillary node | No |
| 68352T006 | | | | | | |
| 75319T003 | 30 | M | W | MET | Skin, back | Dabrafenib, Trametnib |
| 75664T003 | 63 | F | W | MET | Soft tissue | PD1 |
| 75687T001 | 60 | M | W | MET | Metastatic ileo-inguinal node | No |
| 75955T003 | 65 | M | W | MET | Metastatic axillary node | Ipilimumab |
| 76283T003 | 77 | F | U | MET | Metastatic iliac node | No |
| 76484T004 | 61 | M | W | MET | Adrenal gland | Unknown |
| 77956T001 | 71 | M | W | MET | Metastatic axillary node | Unknown |
| 77958T001 | 81 | M | W | MET | Metastatic axillary node | Unknown |

*Unknown

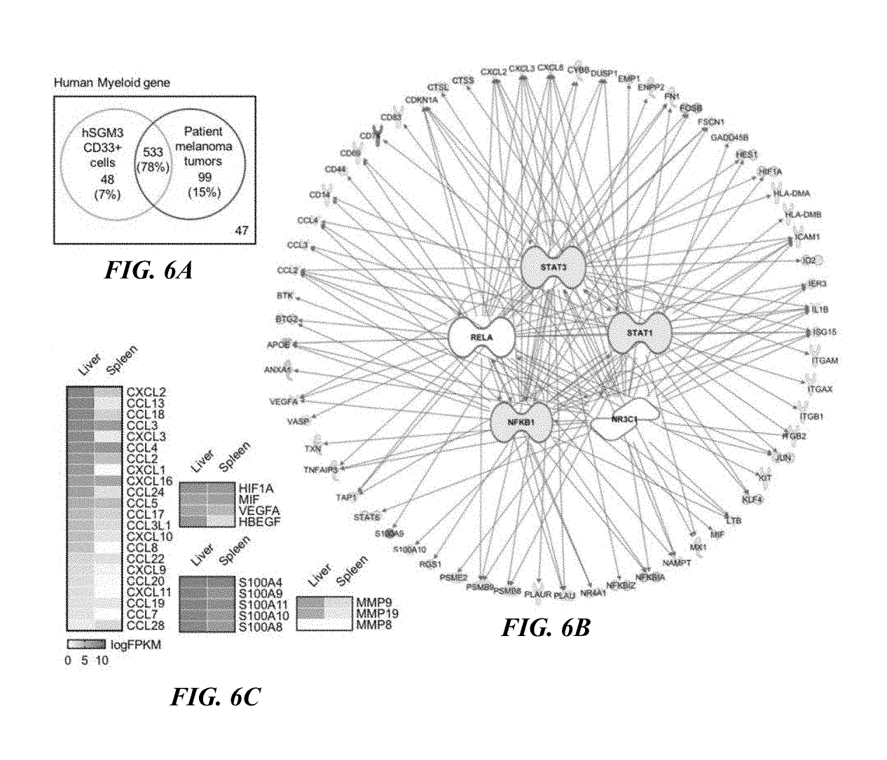

To focus the analysis on the most relevant myeloid genes, we applied gene list validated as myeloid panel for NanoString (727 genes). Generated lists of transcripts were first analyzed separately for hCD33+ from hNSG™-SGM3 mice and for human tumor samples using Ingenuity Pathway Analysis (IPA) to determine the myeloid cell pathways that are enriched and/or altered in both conditions (data not shown). In both cases, the top networks were driven by expression of STAT3, STAT1 and NFKB1 thereby suggesting transcriptional similarity between myeloid cells in both types of samples (data not shown). This was further confirmed by Venn-diagram analysis, which revealed that 78% of transcripts were common between the two sample types with the presence of dominant STAT3, STAT1 and NFKB1-regulated networks (FIG. 6A, 6B, Table 3).

TABLE 3
IPA on upstream regulator for the common myeloid genes expressed in CD33+
cells in hNSG ™-SGM3 mice and human melanoma tumors.

| Upstream | p-value of | |
|---|---|---|
| regulator | overlap | Target molecules in dataset |
| STAT3 | 4.06E−67 | AREG, BATF, BCL2, BCL6, BIRC5, C5AR1, CASP1, CASP7, CCL2, CCL20, |
| | | CCL4, CCL5, CCR1, CCR5, CCRL2, CD209, CD274, CD40, CD74, |
| | | CD80, CD86, CDH1, CDKN1A, CEACAM1, CEBPA, CEBPB, CEBPD, |
| | | CTSL, CXCL10, CXCL2, CXCL3, CXCL8, CXCL9, CXCR3, CXCR4, |
| | | DPP4, EGR2, EGR3, FAS, FASN, FCGR1A, FLT1, FN1, FSCN1, |
| | | FUT4, GATA3, GPR65, HGF, HIF1A, HIST2H2AA3/HIST2H2AA4, |
| | | HLA-DMA, HLA- |
| | | DQA1, ICAM1, ID2, IFIT1, IKBKE, IL10, IL1B, IL1R1, IL1RN, IL4R, IL6, |
| | | IL6R, IRF1, IRF4, IRF5, IRF7, ISG15, ITGAM, ITGB1, ITGB2, JAG1, |
| | | KLF4, LIF, LTA, MAFB, MMP9, MX1, MX2, MYC, MYD88, NAMPT, |
| | | NFATC2, NFKB1, NFKBIZ, NR4A2, PIM2, PLAU, PLAUR, PSMB8, |
| | | PSMB9, PTAFR, PTGS2, S100A9, SERPINB9, SOCS1, SOCS3, STAT1, |
| | | STAT3, TAP1, TGFB1, THBD, TLR3, TNF, TNFRSF1B, TNFSF10, |
| | | USP18, VEGFA |
| RELA | 2.9E−62 | ALCAM, ALOX5AP, APOE, BCL10, BCL2, BIRC2, BIRC3, BIRC5, |
| | | BTG2, C3, CCL19, CCL2, CCL20, CCL22, CCL3, CCL5, CCR7, CD14, |
| | | CD40, CD44, CD69, CD80, CDKN1A, CEBPB, CXCL1, CXCL10, CXCL11, |
| | | CXCL2, CXCL3, CXCL8, CXCL9, CXCR4, CYBB, DUSP1, FAS, |
| | | FN1, FOSB, FSCN1, GCH1, HES1, ICAM1, IER3, IKBKE, IL10, IL15RA, |
| | | IL1B, IL1RN, IL6, IRF1, IRF4, IRF7, ISG15, JUN, KIT, KLF10, LTA, |
| | | LTB, MIF, MMP9, MYC, NAMPT, NFATC1, NFKB1, NFKBIA, |
| | | NFKBIE, NOD2, NR4A1, NR4A2, OLR1, PLAU, PPARG, PSMB9, PTGDS, |
| | | PTGS2, PTX3, SMAD7, STAT5A, TAP1, TAP2, TAPBP, TERF2IP, |
| | | TGFB1, TGM2, TLR2, TNF, TNFAIP3, TNFRSF4, TRAF1, TREM1, VASP, |
| | | VCAM1, VEGFA |
| STAT1 | 3.84E−60 | APOE, BIRC5, C3, C4A/C4B, CASP1, CCL19, CCL2, CCL20, CCL3, |
| | | CCL5, CCR6, CCR7, CCRL2, CD14, CD274, CD40, CD86, CDKN1A, |
| | | CEACAM1, CEBPD, CREM, CSF3R, CTSS, CXCL10, CXCL11, CXCL2, |
| | | CXCL3, CXCL8, CXCL9, CXCR3, DPP4, FAS, FCGR1A, FCGR2B, |
| | | FURIN, GATA3, HAVCR2, HES1, HIF1A, HLA- |
| | | DQA1, ICAM1, IDO1, IFIT1, IL10, IL15, IL15RA, IL1B, IL1R1, IL6, |
| | | IRF1, IRF2, IRF5, IRF7, IRF8, ISG15, ITGAX, JUN, KLF4, MMP9, MX1, |
| | | MYC, PDCD1LG2, PDGFA, PPARG, PSMB8, PSMB9, PSME2, PTGS2, |
| | | S100A10, SMAD2, SMAD7, SOCS1, SOCS3, STAT1, STAT3, TAP1, |
| | | TLR3, TLR4, TLR8, TNF, TNFSF10, TRAF2, TRAFD1, USP18 |
| NFKB1 | 2.5E−54 | APOE, BCL2, BIRC3, BTG2, CCL19, CCL2, CCL20, CCL22, CCL4, |
| | | CCL5, CD40, CD80, CD86, CDKN1A, CSF1, CXCL10, CXCL11, CXCL2, |
| | | CXCL3, CXCL8, CXCL9, CXCR5, CYBB, DUSP1, ENPP2, FAS, FOSB, |
| | | FSCN1, GATA3, GNAI3, HLA-DMB, |
| | | ICAM1, ICOSLG/LOC102723996, IER3, IKBKE, IL10, IL18, |
| | | IL1B, IL1RN, IL6, IRF1, IRF4, ISG15, JAG1, LTA, LTB, MMP9, MYC, |
| | | NFATC1, NFKB1, NFKBIA, NOD2, NR4A1, NR4A2, PDGFA, PLAU, |
| | | PTAFR, PTGS2, PTX3, RGS1, STAT1, TGFB1, TLR2, TNF, TNFAIP3, |
| | | TNFRSF4, TNFSF10, TRAF1, TRAF2, VCAM1, VEGFA |
| NR3C1 | 2.5E−54 | ALOX5AP, ANXA1, APOE, BCL10, BCL2, BCL6, BIRC2, BIRC3, BTK, |
| | | C4A/C4B, CASP10, CASP7, CCL2, CCL4, CCL5, CD38, CD47, CD69, |
| | | CD70, CD83, CDKN1A, CEBPA, CEBPB, CXCL10, CXCL2, CXCL3, |
| | | CXCL8, CXCR4, DAXX, DUSP1, EMP1, ENC1, FADD, FN1, GADD45B, |
| | | GATA3, GEM, GPR65, ICAM1, IER3, IL10, IL15, IL15RA, IL18, IL1B, |
| | | IL1RAP, IL3RA, IL6, INSR, IRF1, IRF8, ISG15, ITGB2, JUN, LIF, |
| | | LTB, MALT1, MAP3K14, MMP8, MMP9, MYC, MYD88, NAMPT, |
| | | NFATC1, NFIL3, NFKB1, NFKBIA, NLRP3, NOD2, OLR1, PDCD1LG2, |
| | | PDGFA, PHLDA2, PPARG, PRKCI, PTGS2, RAF1, RIPK2, RUNX2, |
| | | SERPINB9, SGPP1, SMAD1, STAT3, STAT5A, STAT6, TGFB1, |
| | | THBD, TIMP3, TLR1, TLR2, TLR5, TNF, TNFAIP3, TNFAIP6, TNFAIP8, |
| | | TNFRSF12A, TNFRSF1A, TNFRSF1B, TNFRSF8, TNFRSF9, TNFSF9, |
| | | TRAF1, TUBA4A, TXN, VCAM1 |

Among transcripts present in both types of samples are HIF1a, VEGFA and MIF, well-established tumor-promoting molecules³⁶; as well as transcripts coding for S100 proteins (S100A9 and S100A10), which among other functions regulate myeloid cells^{37, 38} including the immature myeloid cells that contribute to immunosuppressive milieu in cancer^{39, 40} (FIG. 6B). Interestingly, some of the genes that are common between hCD33+ cells from hNSG™-SGM3 mice and patient tumors were not equally represented in hCD33+ cells isolated from liver and spleen (FIG. 6C). Among others, liver hCD33+ cells displayed substantially higher expression transcripts of chemokines that are potent chemoattractant for myeloid cells and which could regulate organ-specific metastasis⁴¹ including CXCL2, CCL13, CCL18 and CXCL3; matrix metalloproteinases that contribute to metastatic process⁴² such as matrix metalloproteinase (MMP) 9 and MMP19; as well as transcripts coding S100 family proteins S1009 and S100A11 (FIG. 6C). Furthermore, while hCD33+ cells from both organs expressed HIF1a, VEGFA and MIF transcripts (FIG. 6c), liver hCD33+ cells uniquely expressed HBEGF transcript coding heparin-binding EGF like growth factor⁴³ suggesting that hCD33+ cells in different organs might utilize different molecular pathways promoting growth of distant tumors. Accordingly, while both liver and spleen hCD33+ cells expressed VEGFA transcript, we found VEGFA protein and its impact on tumor growth only in the spleen (FIGS. 4A-4E).

Finally, tissue analysis showed the close proximity of hCD33+ cells to melanoma cells in the metastatic liver and spleen of hNSG™-SGM3 mice, in a manner similar to that of human melanoma tumor sample (data not shown), and tumor infiltrating CD33+ cells expressed VEGFA in tumors from both hNGS-SGM3 mice and patients (data not shown). Thus, hNSG™-SGM3 mice carry human myeloid cells that are similar to that found in patients and thus these mice resemble the alterations of myeloid cells found in melanoma patients.

### Example 7. Humanized NSG™-SGM3 Mice Support the Development of Metastatic Disease for Breast and Pancreatic Cancer

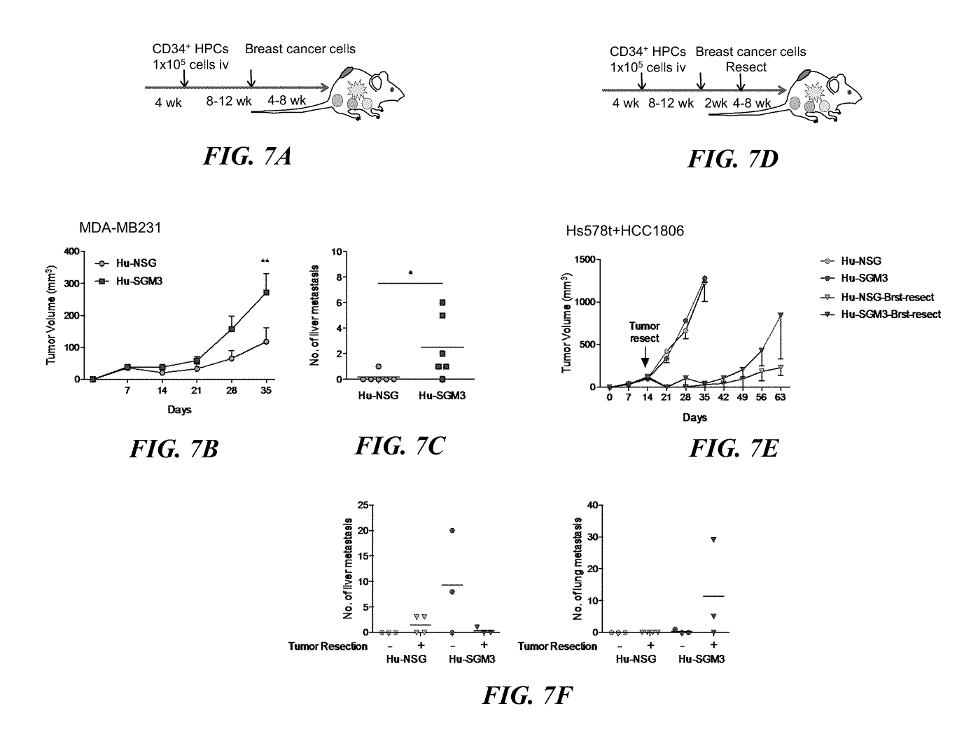

To test if hNSG™-SGM3 mice support metastasis development in other types of cancer, we implanted breast cancer cells in hNSG™ and hNSG™-SGM3 mice (FIG. 7A). We analyzed the hNSG™-SGM3 mice at 5-week post tumor implant and found macroscopic tumors in the liver of hSGM3, but not hNSG™ mice (FIG. 7B). Immunofluorescence analysis of frozen tissue sections of implanted, locally growing tumor and distant liver tumors revealed expression of human cytokeratin 19 (CK19) in the tumor, thus confirming that the tumors are actually breast cancers (data not shown). The analysis of tumor infiltrating leukocytes (TILs) revealed infiltrates with human CD3+ T cells and human CD11c+ myeloid cells secreting IL-1b (data not shown). CK19+ tumor cells could also be found in the mice without macroscopic liver tumors (data not shown). Thus, hNSG™-SGM3 mice support breast cancer metastasis in a process incited by human immune cells. Because the implanted breast tumor grew rapidly at the implantation site thereby limiting the length of follow up and longitudinal studies, we surgically removed the tumor mass at the implantation site 14 days after initial implantation in subsequent experiments (FIG. 7D).

As shown in FIG. 7E, both hNSG™ and hNSG™-SGM3 mice support tumor outgrowth and local recurrence. However, this process was substantially accelerated (by 42 days post-resection) in hSGM3. Surprisingly, we observed a different pattern of metastatic dissemination comparing hNSG™-SGM3 mice with and without surgical intervention (FIG. 7F). In the latter case, we found metastasis in the liver, as described in FIG. 7C. After surgical intervention at the tumor implantation site, we did not observe macroscopic tumors in the liver. We did, however, find tumors in the lungs (FIG. 7F). The presence of human CK19+ breast cancer cells in the lungs was further confirmed by immunofluorescence staining (data not shown).

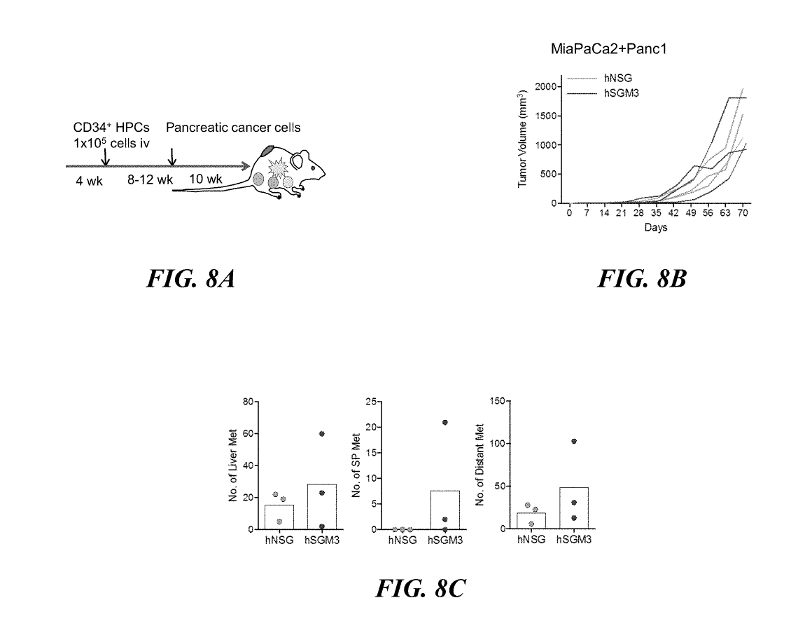

In addition, we also test the capacity of hNSG™-SGM3 to support metastasis of pancreatic cancer (FIG. 8A). We analyzed the hSGM3 mice at 10-week post pancreatic tumor implant (FIG. 8B) and found higher macroscopic tumors in the liver, spleen and combined all distant organs together in hNSG™-SGM3 mice than in hNSG™ mice (FIG. 8C). Thus, hSGM3 facilitate metastatic dissemination for other type of cancer.

Here we show that NSG™-SGM3 mice humanized by transplant of hCD34+ HPCs support development of multi-organ human melanoma metastasis. The process is multifactorial and requires human CD33+ myeloid cells as well as host factors such as human cytokines SCF, GM-CSF and IL-3. The requirement for cell cycle resembles data in patients where increased mitotic rate in the tumor is significantly associated with declining survival rate and can serve as an independent predictor of survival¹⁰. Importantly, the myeloid cells with pro-metastatic activity in our model have similar transcriptional profiles as CD33+ myeloid cells infiltrating metastatic melanoma tumor samples from patients. Indeed, the pathway analysis of RNAseq data from both hNSG™-SGM3⁻ derived CD33+ cells and human melanoma tumor revealed a common upstream regulator STAT3. STAT3 is a well-documented tumor-promoting gene whose activation has been suggested, among others, in melanoma tumorigenesis and metastasis^{44, 45, 46, 47, 48}. In cancer cells, STAT3 activation upregulates c-myc oncogene⁴⁹, MMP2 for tumor invasion⁴⁷, and VEGF expression for tumor angiogenesis⁴⁴. In myeloid cells, STAT3 activation is linked with inhibition of functional maturation of DCs thereby blocking effective anti-cancer T cell immunity^{45, 50}. Furthermore, STAT3-deficient tumor-associated myeloid cells in murine cancer models display diminished capacity to produce VEGF, bFGF, IL-1b, MMP9, CCL2 and CXCL2 that are important for tumor angiogenesis⁵¹. This is consistent with our data on VEGFA expression by the myeloid cells in spleen of hNSG™-SGM3 mice and the decrease in the numbers of spleen melanoma tumors after anti-VEGFA treatment. Thus, our model resembles human disease thereby offering a novel approach to dissecting human cancer/human myeloid cells interface. Indeed, despite the presence of murine myeloid cells, the metastatic dissemination requires human myeloid cells. This suggests that the molecular pathways governing myeloid cell-dependent melanoma metastasis are not available in the absence of human cells. Furthermore, when NSG™ or NSG™-SGM3 mice were adoptively reconstituted with hCD45+ cells isolated from hNSG™-SGM3 mice, only NSG™-SGM3 mice gave rise to distant metastasis. Thus, our data suggest an active mechanism mediated by the host cytokines SCF, GM-CSF, and IL-3 impacting human myeloid cells. Indeed, non-humanized NSG™-SGM3 mice did not support development of distant melanoma tumors thereby excluding the potential direct effects of SCF on melanoma cells via KIT⁵².

At variance with our earlier studies in humanized MISTRG mice where the presence of human myeloid cells accelerated “primary tumors” formed by Me275 melanoma cells implanted SC¹⁸, there was no such effect in hNSG™-SGM3 mice. MISTRG mice carry human CSF-1 (MCSF) and thus support development of human macrophages. Since NSG™-SGM3 mice do not carry human CSF-1 gene, they don't develop bone fide CSF-1 driven human macrophages. These results suggest that different myeloid cell subsets elicit distinct impact on melanoma tumor growth. It will be important to determine whether MISTRG mice support melanoma metastasis in similar manner and whether adding human CSF-1 to NSG™-SGM3 mice will result in a different scenario. In this context, we have not detected brain metastasis; thus, it is possible that these require human CSF-1 dependent cells to occur. Similarly to studies in mouse models, our results suggest that requirements for establishing “primary” tumor are different from those for support of distant tumors⁵³. Various myeloid cells including neutrophils⁵⁴, and myeloid suppressor cells⁵⁵ have been shown to support the tumor growth in distant sites in various cancer types. The governing mechanisms might be direct via cell-cell contact but also indirect as for example in melanoma models where melanoma cell-derived exosomes can deliver signals for angiogenesis and metastatic invasion through bone-marrow derived cells⁵⁶. Tissue stromal cells can also contribute as shown in a recent study of murine B16 melanoma model, where the metastatic niches in the lungs were initiated by tumor exosomal RNA able to stimulate TLR3 on alveolar epithelial cells, which subsequently recruit neutrophils⁵⁷.

Humanized NSG™-SGM3 mice support preferentially human CD4+ T cell development (including cells with the phenotype of regulatory T cells)³³ at the expense of cytotoxic lymphocytes. Furthermore, human T cells are selected in the thymus in the context of mouse MHC^{58, 59, 60}. Thus, the tumors are not exposed to environmental pressure and editing from cytotoxic lymphocytes, which could impact development of distant tumors. Future studies with next generation of NSG™-SGM3 mice engineered to express additional human genes to support development of a more complete human immune system will help address these important questions. It will be particularly interesting to determine, using add-on humanization approach which genes are critical to awaken the non-cycling cells thereby facilitating metastatic dissemination. This could offer novel targets for prevention. Finally, transgenic expression of human cytokines and ensuing high systemic concentrations of cytokines could result in skewed hematopoiesis and exhaustion of HPCs⁶¹ However, it also reflects left shift and emergency hematopoiesis frequently observed in patients⁶². Indeed, the analysis of transcriptional profiles demonstrated nearly 80% overlap between the immune cells in our model and in melanoma patients. Thus, our model complements mouse models of metastatic disease^{63, 64, 65}. It offers a platform for in-depth studies of melanoma, and possibly other tumors, metastatic colonization of distant organs enabling the identification of human-specific molecular pathways.

### Material and Methods

Antibodies and Reagents

Antibodies to human CD14 (MqP9), CD3 (SK7), CD4 (SK3), CD8 (SK1), CD11c (S-HCL-3), CD16 (3G8), CD19 (HIB19), CD20 (2H7), CD33 (P67.6), CD34 (8G12), CD44 (G44-26), CD45 (HI30), CD45RA (HI100), HLA-A2 (BB7.2), HLA-DR (L243) Ki-67 (B56) and mouse CD45 (30-F11), were obtained from BD (Franklin Lakes, N.J.). Antibodies to human CD146 (ME-9F1) were from Mitenyi Biotec. Human HLA-ABC (W6/32) and CD33 (WM53) antibodies from Biolegend was also used. Antibodies to MART-1 (M2-2C10 and M2-9E3) was obtained from Novus Biologicals. Antibodies to human gp100 (NK1-beteb) was from LifeSpan BioSciences and human VEGFA (JH121) antibody was from Thermo Fisher Scientific.

Cell Culture

Melanoma cancer cell lines, Me275, which were established from surgically excised melanoma metastases from patients LAU50 and Me290, were provided by Pedro Romero at the Ludwig Institute for Cancer Research at University of Lausanne (Lausanne, Switzerland); A2058, A375, Colo-829, SKmel5, SKmel24, MDA-MB231, Hs578t, HCC1806, MiaPaCa-2, and Panel were from the American Type Culture Collection (ATCC); WM852 and WM1366 were from Rockland Immunochemicals (Limerick, Pa.). All cell lines were cultured in complete RPMI (RPMI 1640, 25 mM HEPES, 1 mM Sodium Pyruvate, 1% non-essential amino acid, 1% penicillin-streptomycin and 2 mM L-Glutamine) supplemented with 10% FBS at 37° C. with 5% CO2 atmosphere. All cell lines were authentication using Short Tandem Repeat (STR) profiling analysis by ATCC. The mycoplasma test was performed regularly, and all the cell lines were mycoplasma-free for each in vitro and in vivo experiment.

Lentiviral Transduction

The Me275 tumor cell lines were transduced with lentivirus vectors (pLX302-CMV-Luc2) at varying multiplicity of infection (moi) by incubating virions in the culture medium containing 8 g/ml polybrene (Sigma). Stably transduced cells were selected with 3 μg/ml puromycin (In Vivogen). Stable cell line expressing Luc were confirmed by anti-Luc (Luci21 1-107, Novus) intracellular staining and by using luciferin (Xenogen, Alameda, Calif.) and an in vivo imaging system (Xenogen).

Humanized Mice

Humanized mice were generated on parental NSG™ mice or NSG™ carrying SCF, GM-CSF, and IL3 (NSG™-SGM3 or SGM3) obtained from the Jackson Laboratory (Bar Harbor, Me.). Mice were sub-lethally irradiated (10 cGy per gram of body weight) using a ¹³⁷Cs gamma irradiator at the age of four weeks. 100,000 CD34+ HPCs from fetal liver or full-term cord blood (Advanced Bioscience Resources, Alameda Calif.) were given in 200 μL of PBS into the tail-vein intravenously. Mice were bled for the engraftment at 8-12 weeks post HPCs transplant. All protocols were reviewed and approved by the Animal Care and Use Committee at The Jackson Laboratory (Bar Harbor, Me.) and the University of Connecticut Health Center (Farmington, Conn.).

Tumor Model

Tumor cells were from in vitro cultures were injected subcutaneously (SC) into the flanks of the mice. Tumor size was monitored every 7 days with caliper. Tumor volume (ellipsoid) was calculated as follows: (short diameter)2×long diameter/2. Alternatively, luciferase-labeled melanoma cells were injected intravenously into the mice. Mice were killed and the macroscopic metastases were identified and scored in various organs including skin, lymph nodes, liver, spleen, kidney, and lungs. Blood and bone (femur and tibia) were collected for further ex vivo analysis to identify bone metastasis by FACS. For anti-VEGFA (bevacizumab) treatment, mice were given IP anti-VEGFA (Genentech, South San Francisco, Calif.) at 10 mg/kg at day 14 after subcutaneous melanoma cell implantation and 5 mg/kg every 5 days thereafter for a total of 9 treatments. PBS was given intraperitoneally as a vehicle control.

In Vivo Imaging

Before mice were anesthetized with Isoflurnae, an aqueous solution of luciferin (150 mg/kg intraperitoneally) was injected 10 min prior to imaging. The animals were placed into the light-tight chamber of the CCD camera system (Xenogen) and the photons emitted from the luciferase-expressing cells within the animal were quantified for 5 min using the software program Living Image (Perkin Elmer). To image dissected organs, mice were first injected intraperitoneally with luciferin (150 mg/kg) for 3 mins and quickly killed to remove each organ. Organs were placed in the 12-w culture dish with PBS containing 300 μg/ml of luciferin and imaged the same way as previously described with the in vivo imaging system.

Circulating Tumor Cells Analysis

Mice were euthanized and blood was collected with heparin. Cells were first treated with both human and murine Fc blocker (BD) and then stained on ice with mouse CD45 and human CD45, HLA-ABC, CD44, and CD146 antibodies. After washing twice with PBS, the samples were acquired on a LSRII or FACSARIA II (BD Biosciences), and analyzed with FlowJo software (Tree Star, Ashland, Oreg.). Melanoma cells were gated as mouse CD45− and human CD45−, HLA-ABC+, CD44+, CD146+ cells. To isolate CTCs, mCD45−HLA-ABC+CD44+CD146+ cells were sorted by FACSaria.

Immunohistofluorescence Staining

Tissues were embedded in OCT (Sakura Finetek U.S.A., Torrance, Calif.) and snap frozen in liquid nitrogen. Frozen sections were cut at 6 μm, air dried on Superfrost plus slides and fixed with cold acetone for five minutes. Tissue sections were first treated with Background Buster and Fc Receptor Block (Innovex Bioscience, Richmond, Calif.). The sections were then stained with primary mouse monoclonal Abs for one hour at room temperature, followed by staining with fluorochrome-labeled secondary Abs. Respective isotype Abs were used as the control. Finally, sections were counterstained with 1 μg/ml of DAPI (Invitrogen), mounted with Fluoromount, and visualized using a Zeiss Axio fluorescence microscope with ZEN software (Germany) or a Leica SP 8 confocal microscope with Leica LAS AF 2.0 software (Buffalo Grove, Ill.).

Human Immune Cell Purification and Adoptive Transfer

At 14-16-week post-transplant, humanized NSG™-SGM3 mice were euthanized and spleen and liver were collected for single cell suspension. Spleen and liver were first digested with 25 μg/ml Liberase (Roche Diagnostics) for 10 min at 37° C.; single cell suspensions were made and the debris was removed by filtering through a 70 m cell strainer. Live cells were isolated using Ficoll-Paque Plus density gradient centrifugation (GE). Human immune cells were enriched using Mouse/Human Chimera isolation kit (StemCell Technologies) following the manufacturer's protocol. For the enrichment CD33+ or CD33− cells, human CD45+ cells were further stained with PE-conjugated CD33 antibodies for 15 mins and enriched by using EasySep PE selection kit (StemCell Technologies). Isolated human immune cells had purity more than 95%. Human immune cells were given by intravenous injection.

RNA-Seq Sample Preparation, Sequencing and Analysis

Total RNA was isolated from snap frozen metastatic melanoma tissues (Cooperative Human Tissue Network, Pennsylvania) and CD33+ cells from the bone marrow, spleen, and liver of hNSG™-SGM3 mice using an RNA isolation kit following the manufacturer protocol (Qiagen). Total RNA isolated from melanoma cells was run on a NanoDrop 8000 (Thermo Scientific) and a Bioanalyzer 2100 Nano Chip (Agilent Technologies) to determine RNA quantity and quality. Sequencing libraries for the whole transcriptome analysis were prepared using KAPA Stranded mRNA-Seq kit (Roche) according to manufacturer's instruction. First, poly A RNA was isolated from 300 ng total RNA using oligo-dT magnetic beads. Purified RNA was then fragmented at 85° C. for 6 mins, targeting fragments range 250-300 bp. Fragmented RNA were then reverse transcribed with an incubation of 25° C. for 10 mins, 42° C. for 15 mins and an inactivation step at 70° C. for 15 mins. This was followed by second strand synthesis at 16° C., 60 mins. Double stranded cDNA fragments were purified using Ampure XP beads (Beckman). The dscDNA were then A-tailed and ligated with Illumina adaptors (IDT). Adaptor-ligated DNA was purified using Ampure XP beads. This is followed by 10 cycles of PCR amplification. The final library was cleaned up using AMpure XP beads. Sequencing was performed on Illumina NextSeq 500 platform generating single end reads of 75 bp. All primary analysis of RNA-Seq was processed using CASAVA pipeline (Illumina, v1.8.2). Sequences were aligned with Bowtie 2 and counts were generated with RSEM using the annotations from Ensembl GRCh37. The files from alignment were converted to BAM format using SAMtools. Raw counts were normalized to log 2 transformed Fragments Per Kilobase of transcript per Million mapped reads (log₂ (FPKM+1)). Ingenuity pathway analysis (Qiagen) was applied to reveal transcriptional networks. CIBERSORT was used to estimate the proportions of diverse immune cell types using the genes that define the signature expression of the immune cell types. We used the default 22 cell types (LM22) provided (71). RNA-seq data have been deposited at the NCBI Sequence Read Archive (SRA) with accession number SRP141256.

Statistical Analysis

Statistical analysis was performed in Prism (GraphPad). Legend is: ****P<0.0001, ***P<0.001, **P<0.01, *P<0.05, ns=not significant. Comparisons between any 2 groups were analyzed using the Mann-Whitney test or two-tailed t-test. Comparisons between any 3 or more groups were analyzed by analysis of variance (ANOVA).

Cell Cycle Analysis

Cells were first treated with both human and murine Fc blocker (BD Bioscience) and then stained on ice with surface antibodies. After washing twice with PBS, the samples were fixed and permeabilized with FOXP3/Transcription Factor Staining Buffer (Thermo Fisher Scientific) following manufacturer protocol. Briefly, cells were fixed with Fixation/Permeabilization buffer at 4° C. for 30 minutes, followed by two washes with Permeabilization buffer and stained with Ki-67 antibody and DAPI for 30 minutes at room temperature. After washing twice with PBS, the samples were acquired on a Symphony A5 (BD Bioscience) and analyzed with FlowJo software (Tree Star).

LDH Measurement

Plasma were collected by centrifuging blood with heparin at 500×g for 10 minutes. LDH were measured with AU680 chemistry analyzer (Beckman Coulter) following the manufacturer protocol.

Histo-Cytometry

In situ quantitative analysis of tissues was based on previously published methodology. Briefly, OCT-embedded tissue sections were first immunofluorescence stained with DAPI, MART-1 (M2-2C10 and M2-9E3)/gpl00 (NK1-beteb), human VEGFA (JH121) and human CD45 (HI-30). Whole tissue scans were acquired using a Leica SP8 confocal microscope with motorized stage for tiled imaging (Leica Microsystems). Each scan was then analyzed with Imaris (Bitplane). Using the “spot” function in Imaris, the images were subdivided into individual cells with a nucleus diameter equal or larger than 6 μm. The accuracy of segmentation was manually verified for each sample and adjusted if needed. Finally, for each generated spot, the x and y coordinates and the sum intensity values for all channels were exported into a .fcs file to be visualized and quantified using FlowJo software.

### REFERENCES

1. Lifetime Risk (Percent) of Being Diagnosed with Cancer by Site and Race/Ethnicity: Both Sexes, 18 SEER Areas, 2009-2012 2016.
2. Lifetime Risk (Percent) of Dying from Cancer by Site and Race/Ethnicity: Both Sexes, Total US, 2009-2012. 2014.
3. Melanoma of the Skin (Invasive). 2016/02/20]; Available from: http://seer.cancer.gov/csr/1975_2012/results_merged/sect_16 melanoma_skin.pdf.
4. Dasgupta, T. and R. Brasfield, Metastatic Melanoma. A Clinicopathological Study. Cancer, 1964. 17: p. 1323-39.
5. Einhorn, L. H., et al., Prognostic correlations and response to treatment in advanced metastatic malignant melanoma. Cancer Res, 1974. 34(8): p. 1995-2004.
6. Meyer, J. E. and L. Stolbach, Pretreatment radiographic evaluation of patients with malignant melanoma. Cancer, 1978. 42(1): p. 125-6.
7. Nathanson, L., T. C. Hall, and S. Farber, Biological aspects of human malignant melanoma.

Cancer, 1967. 20(5): p. 650-5.
8. Patel, J. K., et al., Metastatic pattern of malignant melanoma. A study of 216 autopsy cases. Am J Surg, 1978. 135(6): p. 807-10.
9. Khoja, L., et al., Biomarker utility of circulating tumor cells in metastatic cutaneous melanoma. J Invest Dermatol, 2013. 133(6): p. 1582-90.
10. Gil-Bernabe, A. M., et al., Recruitment of monocytes/macrophages by tissue factor-mediated coagulation is essential for metastatic cell survival and premetastatic niche establishment in mice. Blood, 2012. 119(13): p. 3164-75.
11. Mestas, J. and C. C. Hughes, Of mice and not men: differences between mouse and human immunology. J Immunol, 2004. 172(5): p. 2731-8.
12. Rongvaux, A., et al., Human hemato-lymphoid system mice: current use and future potential for medicine. Annu Rev Immunol, 2013. 31: p. 635-74.
13. Sancho, D. and C. Reis e Sousa, Sensing of cell death by myeloid C-type lectin receptors. Curr Opin Immunol, 2013. 25(1): p. 46-52.
14. Brown, G. D. and S. Gordon, Immune recognition: A new receptor for beta-glucans. Nature, 2001. 413: p. 36-7.
15. Kanazawa, N., Dendritic cell immunoreceptors: C-type lectin receptors for pattern-recognition and signaling on antigen-presenting cells. J Dermatol Sci, 2007. 45(2): p. 77-86.
16. Williams, A., R. A. Flavell, and S. C. Eisenbarth, The role of NOD-like Receptors in shaping adaptive immunity. Curr Opin Immunol, 2010. 22(1): p. 34-40.
17. Traggiai, E., et al., Development of a human adaptive immune system in cord blood cell-transplanted mice. Science, 2004. 304(5667): p. 104-7.
18. Matsumura, T., et al., Functional CD5+ B cells develop predominantly in the spleen of NOD/SCID/gammac(null) (NOG) mice transplanted either with human umbilical cord blood, bone marrow, or mobilized peripheral blood CD34+ cells. Exp Hematol, 2003. 31(9): p. 789-97.
19. Willinger, T., et al., Improving human hemato-lymphoid-system mice by cytokine knock-in gene replacement. Trends Immunol, 2011. 32(7): p. 321-7.
20. Willinger, T., et al., Human IL-3/GM-CSF knock-in mice support human alveolar macrophage development and human immune responses in the lung. Proc Natl Acad Sci USA, 2011. 108(6): p. 2390-5.
21. Rathinam, C., et al., Efficient differentiation and function of human macrophages in humanized CSF-1 mice. Blood, 2011. 118(11): p. 3119-28.
22. Rongvaux, A., et al., Development and function of human innate immune cells in a humanized mouse model. Nat Biotechnol, 2014. 32(4): p. 364-72.
23. Billerbeck, E., et al., Development of human CD4+FoxP3+ regulatory T cells in human stem cell factor-, granulocyte-macrophage colony-stimulating factor-, and interleukin-3-expressing NOD-SCID IL2Rgamma(null) humanized mice. Blood, 2011. 117(11): p. 3076-86.
24. Balch, C. M., et al., Final version of 2009 AJCC melanoma staging and classification. J Clin Oncol, 2009. 27(36): p. 6199-206.
25. Hodis, E., et al., A landscape of driver mutations in melanoma. Cell, 2012. 150(2): p. 251-63.
26. Robert, C., et al., Anti-programmed-death-receptor-1 treatment with pembrolizumab in 50 ipilimumab-refractory advanced melanoma: a randomised dose-comparison cohort of a phase 1 trial. Lancet, 2014. 384(9948): p. 1109-17.
27. Brehm, M A., et al., Human immune system development and rejection of human islet allografts in spontaneously diabetic NOD-Rag1null IL2rgammanull Ins2Akita mice. Diabetes, 2010, 59: 2265-2270.
28. Huntington, N D., et al., IL-15 trans-presentation promotes human NK cell development and differentiation in vivo. J. Exp. Med., 2009, 206: 25-34.
29. Melkus M W, et al. Humanized mice mount specific adaptive and innate immune responses to EBV and TSST-1. Nat Med, 2006, 12: 1316-1322.
30. Shultz L D, et al. Generation of functional human T-cell subsets with HLA-restricted immune responses in HLA class I expressing NOD/SCID/IL2r gamma(null) humanized mice. Proceedings of the National Academy of Sciences of the United States of America, 2010 107: 13022-13027.
31. Yu C I, et al. Broad influenza-specific CD8+ T-cell responses in humanized mice vaccinated with influenza virus vaccines. Blood, 2008, 112: 3671-3678.
32. Aspord C, et al. Breast cancer instructs dendritic cells to prime interleukin 13-secreting CD4+ T cells that facilitate tumor development. J Exp Med, 2007, 204: 1037-1047.
33. Pedroza-Gonzalez A, et al. Thymic stromal lymphopoietin fosters human breast tumor growth by promoting type 2 inflammation. J Exp Med, 2011, 208: 479-490.
34. Wu T C, et al. Reprogramming tumor-infiltrating dendritic cells for CD103+CD8+ mucosal T-cell differentiation and breast cancer rejection. Cancer Immunol Res, 2014, 2: 487-500.
35. Traggiai E, et al. Development of a human adaptive immune system in cord blood cell-transplanted mice. Science, 2004, 304: 104-107.
36. Matsumura T, et al. Functional CD5+ B cells develop predominantly in the spleen of NOD/SCID/gammac(null) (NOG) mice transplanted either with human umbilical cord blood, bone marrow, or mobilized peripheral blood CD34+ cells. Experimental hematology, 2003, 31: 789-797.
37. Willinger T, et al. Human IL-3/GM-CSF knock-in mice support human alveolar macrophage development and human immune responses in the lung. Proceedings of the National Academy of Sciences of the United States of America, 2011, 108: 2390-2395.
38. Rathinam C, et al. Efficient differentiation and function of human macrophages in humanized CSF-1 mice. Blood, 2011, 118: 3119-3128.
39. Nicolini F E, et al. NOD/SCID mice engineered to express human IL-3, GM-CSF and Steel factor constitutively mobilize engrafted human progenitors and compromise human stem cell regeneration. Leukemia, 2004, 18: 341-347.
40. Wunderlich M, et al. AML xenograft efficiency is significantly improved in NOD/SCID-IL2RG mice constitutively expressing human SCF, GM-CSF and IL-3. Leukemia, 2010 24: 1785-1788.
41. Billerbeck E, et al. Development of human CD4+FoxP3+ regulatory T cells in human stem cell factor-, granulocyte-macrophage colony-stimulating factor-, and interleukin-3-expressing NOD-SCID IL2Rgamma(null) humanized mice. Blood, 2011, 117: 3076-3086.
42. Agarwala S S, et al. LDH correlation with survival in advanced melanoma from two large, randomised trials (Oblimersen GM301 and EORTC 18951). Eur J Cancer, 2009, 45: 1807-1814.
43. Liang W C, et al. Cross-species vascular endothelial growth factor (VEGF)-blocking antibodies completely inhibit the growth of human tumor xenografts and measure the contribution of stromal VEGF. J Biol Chem, 2006, 281: 951-961.
44. Hu Z, et al. A compact VEGF signature associated with distant metastases and poor outcomes. BMC Med, 2009, 7: 9.
45. Bertheloot D, Latz E. HMGB1, IL-1alpha, IL-33 and S100 proteins: dual-function alarmins. Cell Mol Immunol, 2017: 14: 43-64.
46. Xia C, et al. S100 Proteins As an Important Regulator of Macrophage Inflammation. Front Immunol, 2017, 8: 1908.
47. Ostrand-Rosenberg S, Sinha P. Myeloid-derived suppressor cells: linking inflammation and cancer. Journal of immunology, 2009, 182: 4499-4506.
48. Tcyganov E, et al. Plasticity of myeloid-derived suppressor cells in cancer. Curr Opin Immunol, 2018, 51: 76-82.
49. Zlotnik A, et al. Homeostatic chemokine receptors and organ-specific metastasis. Nat Rev Immunol, 2011, 11: 597-606.
50. Sternlicht M D, Werb Z. How matrix metalloproteinases regulate cell behavior. Annu Rev Cell Dev Biol 17, 463-516 (2001).
51. Miyamoto S, et al. Heparin-binding epidermal growth factor-like growth factor as a new target molecule for cancer therapy. Adv Exp Med Biol, 2008, 622: 281-295.
52. Niu G, et al. Constitutive Stat3 activity up-regulates VEGF expression and tumor angiogenesis. Oncogene, 2002, 21: 2000-2008.
53. Wang T, et al. Regulation of the innate and adaptive immune responses by Stat-3 signaling in tumor cells. Nat Med, 2004, 10: 48-54.
54. Xie T X, et al. Activation of stat3 in human melanoma promotes brain metastasis. Cancer Res, 2006, 66: 3188-3196.
55. Xie T X, et al. Stat3 activation regulates the expression of matrix metalloproteinase-2 and tumor invasion and metastasis. Oncogene, 2004, 23: 3550-3560.
56. Niu G, et al. Roles of activated Src and Stat3 signaling in melanoma tumor cell growth. Oncogene, 2002, 21: 7001-7010.
57. Kiuchi N, et al. STAT3 is required for the gpl30-mediated full activation of the c-myc gene. J Exp Med, 1999, 189: 63-73.
58. Sumimoto H, et al. The BRAF-MAPK signaling pathway is essential for cancer-immune evasion in human melanoma cells. J Exp Med, 2006, 203: 1651-1656.
59. Kujawski M, et al. Stat3 mediates myeloid cell-dependent tumor angiogenesis in mice. J Clin Invest, 2008, 118: 3367-3377.
60. Huang S, et al. Enforced c-KIT expression renders highly metastatic human melanoma cells susceptible to stem cell factor-induced apoptosis and inhibits their tumorigenic and metastatic potential. Oncogene, 1996, 13: 2339-2347.
61. Gupta G P, et al. Mediators of vascular remodelling co-opted for sequential steps in lung metastasis. Nature, 2007, 446: 765-770.
62. Park J, et al. Cancer cells induce metastasis-supporting neutrophil extracellular DNA traps. Sci Transl Med, 2016, 8: 361ra138.
63. Kumar V, et al. The Nature of Myeloid-Derived Suppressor Cells in the Tumor Microenvironment. Trends in immunology, 2016, 37: 208-220.
64. Peinado H, et al. Melanoma exosomes educate bone marrow progenitor cells toward a pro-metastatic phenotype through MET. Nat Med, 2012, 18: 883-891.
65. Liu Y, et al. Tumor Exosomal RNAs Promote Lung Pre-metastatic Niche Formation by Activating Alveolar Epithelial TLR3 to Recruit Neutrophils. Cancer Cell, 2016, 30: 243-256.
66. Halkias J, et al. Conserved and divergent aspects of human T-cell development and migration in humanized mice. Immunol Cell Biol, 2015, 93: 716-726.
67. Plum J, et al. Human intrathymic development: a selective approach. Semin Immunopathol, 2008, 30: 411-423.
68. Saito Y, et al. The in vivo development of human T cells from CD34(+) cells in the murine thymic environment. Int Immunol, 2002, 14: 1113-1124.
69. Beyer A I, Muench M O. Comparison of Human Hematopoietic Reconstitution in Different Strains of Immunodeficient Mice. Stem Cells Dev, 2017, 26: 102-112.
70. Schmidt H, et al. Elevated neutrophil and monocyte counts in peripheral blood are associated with poor survival in patients with metastatic melanoma: a prognostic model. Br J Cancer, 2005, 93: 273-278.
71. McAllister S S, Weinberg R A. The tumour-induced systemic environment as a critical regulator of cancer progression and metastasis. Nat Cell Biol, 2014, 16: 717-727.
72. Massague J, Obenauf A C. Metastatic colonization by circulating tumour cells. Nature, 2016, 529: 298-306.
73. Lambert A W, et al. Emerging Biological Principles of Metastasis. Cell, 2017, 168: 670-691.
74. Gerner M Y, et al. Histo-cytometry: a method for highly multiplex quantitative tissue imaging analysis applied to dendritic cell subset microanatomy in lymph nodes. Immunity, 2012, 37: 364-376.
75. Kim D, et al. TopHat2: accurate alignment of transcriptomes in the presence of insertions, deletions and gene fusions. Genome Biol, 2013, 14: R36.
76. Anders S, Pyl P T, Huber W. HTSeq—a Python framework to work with high-throughput sequencing data. Bioinformatics, 2015, 31:166-169.
77. Harrow J, et al. GENCODE: the reference human genome annotation for The ENCODE Project. Genome Res, 2012, 22: 1760-1774.
78. Li H, et al. The Sequence Alignment/Map format and SAMtools. Bioinformatics, 2009, 25: 2078-2079.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. A method of assessing properties and/or pathways of human cancer cells, the method comprising:
delivering to a transgenic humanized NSG™-SGM3 mouse human cancer cells, and
assessing one or more properties and/or pathways of the human cancer cells.

2. The method of claim 1, wherein the transgenic humanized NSG™-SGM3 mouse comprises human CD34+ hematopoietic progenitor cells.

3. The method of claim 1, wherein the human cancer cells are selected from human melanoma cells, human breast cancer cells, and human pancreatic cancer cells.

4. The method of claim 3, wherein the human cancer cells are metastatic human melanoma cells.

5. The method of claim 3, wherein the human cancer cells are non-metastatic human melanoma cells.

6. The method of claim 1, wherein the step of assessing comprises assessing metastasis of the human cancer cells to distant organs of the transgenic humanized NSG™-SGM3 mouse.

7. The method of claim 1, wherein the step of assessing comprises assessing biochemical properties, physiological properties, metabolic processes, signaling pathways, life cycle, and/or chemical composition of the human cancer cells.

8. The method of claim 1, wherein the method further comprises delivering to the transgenic humanized NSG™-SGM3 a therapeutic agent, optionally a monoclonal antibody.

9. The method of 8, wherein the method further comprises assessing the effects of the therapeutic agent.

10. A method of producing a transgenic humanized mouse model of metastatic cancer , comprising delivering to a the transgenic humanized NSG™-SGM3 mouse metastatic human cancer cells to produce a metastatic cancer mouse model comprising tumor nodules in organs of the mouse model, wherein the tumor nodules comprise metastatic human cancer cells, optionally wherein the cancer is melanoma.

11. The method of claim 10, the transgenic humanized NSG™-SGM3 mouse comprises human CD34⁺ hematopoietic progenitor cells.

12. The method of claim 10, wherein the metastatic cancer mouse model comprises human CD146+ circulating tumor cells, human CD45+ immune cells, and/or human CD33+ myeloid cells, optionally wherein the CD33+ myeloid cells express cytokine receptors for SCF, cytokine receptors for GM-CSF, and/or cytokine receptors for IL-3.

13. The method of claim 10, wherein the metastatic human cancer cells are selected from A375, A2058, and Me275 cells.

14. The method of claim 10, wherein 1×10³ to 1×10⁸ metastatic human cancer cells are delivered to the transgenic humanized NSG™-SGM3 mouse.

15. The method of claim 10, wherein the metastatic human cancer cells are delivered subcutaneously.

16. The method of claim 10, wherein the tumor nodules are present in the mouse liver and/or spleen.

17. The method of claim 10, wherein the level of serum lactate dehydrogenase (LDH) in the metastatic cancer mouse model is elevated relative to a control transgenic NSG™ mouse that does not comprise the metastatic human cancer cells.

18. A transgenic NSG™-SGM3 mouse comprising human CD34+ hematopoietic progenitor cells and human melanoma cells.

19.-23. (canceled)

24. The method of claim 10, wherein the cancer is breast cancer.

25. The method of claim 10, wherein the cancer is pancreatic cancer.
